(19) United States
(12) Patent Application Publication
(10) Pub. No.: US 2024/0033189 A1
(43) Pub. Date: Feb. 1, 2024

(54) METHOD FOR PRODUCING A PERSONALIZED COSMETIC PREPARATION

(71) Applicant: BASF SE, Ludwigshafen am Rhein (DE)

(72) Inventors: Myriam SOHN, Grenzach-Wyhlen (DE); Stanislaw KRUS, Grenzach-Wyhlen (DE); Anja SUCKERT, Dusseldorf-Holthausen (DE)

(21) Appl. No.: 18/010,220

(22) PCT Filed: Jun. 17, 2021
(86) PCT No.: PCT/EP2021/066430

§ 371 Date: Jul. 27, 2023

(30) Foreign Application Priority Data
Jun. 18, 2020 (EP) 20180895.3

(51) Int. Cl. A61K 8/02; A61K 8/37; A61Q 17/04; A61Q 19/08; B01F 33/84

(52) U.S. Cl. CPC A61K 8/02; A61K 8/37; A61Q 17/04; A61Q 19/08; B01F 33/844; A61K 2800/596; A61K 2800/48; A61K 2800/522; A61K 2800/43; A61K 2800/87; A61K 2800/882; B01F 2101/21

(57) ABSTRACT

The invention lies in the field of personalized sunscreens, the composition of which is partly determined (personalized) by the end user, and relates to a method for production thereof, and to the use of cosmetic mixtures in the dispensing vessels for performance of the method.

The invention lies in the field of personalized sunscreens, the composition of which is partly determined (personalized) by the end user, and relates to a method for production thereof, and to dispensing vessels for production thereof, and to the use of cosmetic mixtures in the dispensing vessels, or to the dispensing vessels thereof for performance of the method.

A multitude of sunscreens are available to the end user, which fulfill most demands and wishes from the end users on the effect thereof in a standard manner. For example, there are day creams comprising not only UV filters but additionally nourishing skin lotions that also have a cooling effect and are perfume-free. But there are also end users who, on account of peculiarities of their skin or hair or on account of environmental dispositions or other wishes, would prefer to use only personally tailored (“personalized”) sunscreens or would like to partly determine the composition of the cosmetics. In particular, every skin has a different need for protection from UV radiation.

EP-A-00443741 describes an apparatus for production of personalized hair products, wherein the end user is able to produce these themselves at the juncture of use. For instance, it is thus possible to freshly produce permanent wave compositions. For this purpose, end user data such as hair length, hair condition or the like are input into a computer which passes on instructions for creation of the formulation to the apparatus and for this purpose mixes the necessary amounts of components with one another from reservoir vessels.

According to EP1656853, cosmetics for the skin that are personalized by the end user are producible to a certain degree, in that 2 different cosmetic compositions are stored in separate chambers and the user is able to vary the ratio of the two mixtures to one another via actuation of a control element, with the intention of achieving different optical effects such as gloss or the like.

EP-B-2038189 in turn describes a method in which the end user is able to produce domestically customary amounts of cosmetics from exactly premeasured ingredients in packaging units such as capsules, by first directing heated water into a chamber comprising an oily phase. The mixture is then directed onward to a homogenizing unit for formation of an emulsion. For cosmetic formulations having multiple cosmetic ingredients, this method produces emulsions in successive production steps, with the need to store the different raw materials separately. If the use of just one capsule is desired, the capsule has to be divided in order that the ingredients are stored separately.

International patent application WO2018/073541 discloses an apparatus for production of cosmetic products having a water tank and a movable plate that can accept 2 or more capsules, wherein texturing agents are present in one capsule and other active constituents in the other capsule(s).

International patent application WO2018/073541 proposes working with different capsules, wherein the texturing agent is always in one capsule, preferably a cosmetic oil, optionally together with an emulsifier based on amphiphilic molecules. Alternatively, the texturing agent may be a polymer, for example a polysaccharide such as xanthan gum or an acrylic acid-based polymer. The cosmetically active ingredient is to be present in the second capsule, for example antiwrinkle compositions or moisturizers, optionally together with a preservative. Perfuming agents may be present in a further capsule. No specific formulations are disclosed in the application.

For the production of domestically customary amounts of sunscreens by or for the end user in situ, it is not possible to copy the industrial scale processes with a complicated process regime and temperature regime; instead, simple and rapid production processes are needed. However, sunscreens based on water and oil or comprising sparingly soluble ingredients such as pigments, UV filters, perfumes etc. are comparatively difficult to produce since ingredients and active ingredients that are barely miscible or immiscible cannot easily be converted to stable, finely divided emulsions. The various “personalized” active ingredients that may be present in the cosmetic formulations according to the demand or wish of the end user can also complicate the formation and stability of the cosmetic formulations.

Accordingly, it was an object of the present invention to provide a simple method for production of sunscreens by the end user in domestically customary amounts, in which the end user partly decides the composition of the sunscreens according to their wishes/demands or requirements. The method is especially to be suitable for sunscreens that are based on water and oil or for such sunscreens that comprise water- or oil-insoluble active ingredients. In addition, the method is to make available to the end user homogeneous sunscreens that enable a high degree of personalization, i.e. are suitable for many active cosmetic ingredients, and permit a personalized SPF (sun protection factor). Finally, the method is to afford sunscreens that are perceived by the end user to be comparable in terms of quality to cosmetic formulations produced on an industrial scale, i.e., for example, are homogeneous and storage-stable over a long period of time.

The object of the present invention was achieved by a method of producing a sunscreen in domestically customary amounts, controllable by and/or according to the wishes of the end user of the sunscreen (“personalization”), in a device having a mixing device a) and at least one dispensing device b) equipped with at least 2 dispensing vessels, in which the base mixture is present in one of the dispensing vessels b1) and the active cosmetic ingredients suitable for personalization in the other of the dispensing vessels b2), in which the contents of the dispensing vessels b1) and b2) are emptied into the mixing device a) and mixed in the mixing device, wherein the contents of the dispensing vessel b1) comprising the base mixture are emptied into the mixing device together with at least one emollient, at least one interface-active substance and at least one UV filter, wherein the contents of the dispensing vessel b1) also comprise at least one rheology modifier or no rheology modifier.

In the context of the present invention, the term “sunscreen” is understood to mean a cosmetic which is used for UV protection of the end user's skin and hair. A sunscreen may also be used for daycare, providing UV protection for the skin and/or hair. The invention relates, for example, to sunscreens selected from gels, gel creams, milks, hydroformulations, cosmetic oils and oil gels, face care products, personal care products. In the context of the invention, the terms “sunscreen” and “cosmetic formulation” are interchangeable. According to the invention, the sunscreen is personalized and may therefore, as well as a personalized sun protection factor, also include further personalized properties, such as odor or sensory properties.

Further sunscreens of the invention are skin cosmetic formulations, in particular those for additional care of the skin. These especially take the form of W/O or preferably O/W skin creams, day creams, eye creams, face creams, antiwrinkle creams, mimic creams, humectant creams, bleaching creams, vitamin creams, skin lotions, care lotions, milks, balsams, gels and humectant lotions, humectant emulsions, face creams, body creams and hand creams.

Further cosmetic formulations of the invention are antiacne products, repellents and babycare products.

The sunscreens produced in accordance with the invention may take the form of creams, gels, lotions, milk, sera, fluids, alcoholic and aqueous/alcoholic solutions, emulsions, wax/oil masses, pastes or ointments.

In the context of the present invention, the term “domestically customary amount” is understood to mean an amount which is required by the end user at least for one cosmetic application and is not more than the amount of sunscreen that the end user will typically purchase commercially.

These amounts will vary depending on the sunscreen, but are typically in the region of at least a few ml or mg, usually 1 ml or mg, and a maximum of 1000 ml or 1000 g. More preferably, in the context of the invention, sunscreens are produced in amounts of 15 to 50 ml or g, preferably 20 to 35 ml or g, especially 30 ml or g.

In the context of the present invention, the term “end user” is understood to mean the consumer who uses or would like to use the sunscreen.

In the context of present invention, the term “dispensing vessel” is understood to mean a vessel from which the mixture or active ingredient present is dispensed.

In the context of the present invention, the expression “active cosmetic ingredients for personalization” is understood to mean substances that are present in the sunscreen on account of the personal (individual) demands of the end user on the effect of the sunscreen, for example anti-aging, humectant, specific perfume, UV protection (SPF), specific sensory properties, etc. Such active cosmetic ingredients for personalization will also be referred to hereinafter merely as “active cosmetic ingredients” for short.

In the context of the present invention, the term “emollient” is understood to mean substances that make the skin soft and supple, especially by supplying the skin with lipids or reducing evaporation or increasing the moisture content of the skin. Suitable emollients are substances from the group of the oils, fats, waxes, hydrocarbons and/or organosilicon compounds that are liquid at room temperature or have a melting point <45° C.

In the context of the present invention, the term “interface-active substances” is understood to mean compounds that lower the interfacial tension of a liquid or interfacial tension between two phases. Such compounds are also referred to as surfactants or emulsifiers.

In the context of present invention, the term “rheology modifier” is understood to mean substances that alter the deformation and flow properties of material. This comprises organic or inorganic compounds, usually macromolecules, which alter the intermolecular forces through formation of cohesion (intramolecular) or adhesion (intramolecular) in such a way that the viscosity of the coherent phase of the cosmetic formulation is preferably increased.

In the context of the present invention, the method of the invention is conducted with a dispensing vessel b1) comprising the base mixture and, wherein the base mixture comprises at least one emollient, an interface-active substance, a UV filter and a rheology modifier or no rheology modifier.

Emollient

Emollients present in the base mixture may be oils, fats and/or waxes, for example from the group formed by investors, wax esters, waxes, triglycerides or partial glycerides, natural vegetable oils or fats, hydrocarbons, organosilicon compounds, Guerbet alcohols, mono-/dialkyl ethers, mono-/dialkyl carbonates, and mixtures thereof.

From the group of the esters, examples of esters that may be present include those of linear fatty acids with linear or branched fatty alcohols, esters of linear fatty alcohols with linear or branched carboxylic acids, esters of alkylhydroxycarboxylic acids with linear or branched fatty alcohols, esters of linear or branched fatty acids with polyhydric alcohols such as diols or trimer triol, wax esters, triglycerides or partial glycerides (called mono-/di-/triglyceride esters), esters of fatty alcohols and/or Guerbet alcohols with aromatic carboxylic acids, esters of dicarboxylic acids with linear or branched alcohols, natural vegetable oils or fats and mixtures thereof.

Suitable examples from the group of esters of linear C₆-C₂₂ fatty acids with linear or branched C₆-C₂₂ fatty alcohols or esters of branched C₆-C₂₂ carboxylic acids with linear or branched C₆-C₂₂ fatty alcohols are myristyl myristate (Cetiol® MM), myristyl isostearate, myristyl oleate, myristyl erucate, cetyl isostearate, cetyl oleate, cetyl erucate, stearyl myristate, stearyl isostearate, stearyl oleate, stearyl erucate, isostearyl myristate, isostearyl palmitate, isostearyl stearate, isostearyl isostearate, isostearyl oleate, Isopropyl Myristate, Isopropyl Palmitate, oleyl myristate, oleyl palmitate, oleyl stearate, oleyl isostearate, oleyl oleate, oleyl behenate, oleyl erucate (Cetiol® J 600), behenyl oleate, behenyl erucate, erucyl myristate, erucyl palmitate, erucyl stearate, erucyl isostearate, erucyl oleate, erucyl behenate and erucyl erucate, Ethylhexyl Stearate (Cetiol® 868), Hexyl Laurate (Cetiol® A), Coco-Caprylate (Cetiol® C5), Coco-Caprylate/Caprate (Cetiol® LC, Cetiol® C 5C), Propylheptyl Caprylate (Cetiol® Sensoft), Cetearyl Isononanoate (Cetiol® SN), Decyl Oleate (Cetiol® V), Cetearyl Ethylhexanoate.

Additionally suitable are esters of alkylhydroxycarboxylic acids with linear or branched C₆-C₂₂ fatty alcohols, preferably esters of lactic acid such as lauryl lactate.

Additionally suitable are esters of dicarboxylic acids and linear or branched alcohols, preferably esters of malic acid, adipic acid and/or sebacic acid, such as dibutyl adipate, dioctyl malate and/or diisopropyl sebacate.

Also suitable are esters of linear and/or branched fatty acids with polyhydric alcohols (such as propylene glycol, dimer diol or trimer triol), such as Propylene Glycol Dicaprylate/Dicaprate (Myritol® PGDC), triglycerides based on C₆-C₁₀ fatty acids, liquid mono-/di-/triglyceride mixtures based on C₆-C₁₈ fatty acids (Myritol® 331, Myritol® 312, Myritol® 318), esters of C₆-C₂₂ fatty alcohols and/or Guerbet alcohols with aromatic carboxylic acids, especially benzoic acid, esters of benzoic acid with linear and/or branched C₆-C₂₂ alcohols (e.g. Finsolv® TN, Cetiol® AB).

Of good suitability as natural, especially vegetable, fats and oils are groundnut oil, soybean oil, jojoba oil, rapeseed oil, avocado oil, argan oil, castor oil, sunflower oil, palm oil, palm kernel oil, linseed oil, almond oil, wheat germ oil, macadamia nut oil, olive oil, sesame oil, cocoa butter and shea butter, for example Cegesoft® PFO, Cegesoft® PS 6, Cegesoft® SBE, Cegesoft® SH, Cegesoft® VP or Cetiol® SB 45.

Also usable as emollient are, for example, natural vegetable waxes such as fruit waxes (for example orange waxes) and animal waxes such as wool wax.

Also suitable as emollient are C₁₂-C₁₅ fatty alcohols that are usually obtained from natural fats, oils and waxes, such as lauryl alcohol, myristyl alcohol or 1-pentadecanol.

Further suitable emollients are organosilicon compounds, which are frequently referred to simply as silicones. They may take the form of cyclic, branched or linear silicones. Silicones are high molecular weight synthetic polymeric compounds in which silicon atoms are joined via oxygen atoms in a chain-like and/or grid-like manner and the remaining valences of silicon are satisfied by hydrocarbon radicals (usually methyl, more rarely ethyl, propyl, phenyl groups etc.). Systematically, the silicones are referred to as polyorganosiloxanes.

Advantageous polyorganosiloxanes are, for example, the methyl-substituted polyorganosiloxanes that can be represented by the following structural formula:

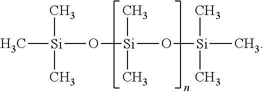

They also referred to as Polydimethylsiloxane or Dimethicone (INCI). Dimethicones come in various chain lengths and with various molecular weights. They are available, for example, under the Abil® 350 trade name from Evonik or Xiameter PMX-200 Silicone Fluid trade name from Dow Chemicals.

Also advantageous are phenylmethylpolysiloxane (INCI: Phenyl Dimethicone, Phenyl Trimethicone), cyclic silicones (e.g. decamethylcyclopentasiloxane or dodecamethylcyclopentasiloxane), which are also referred to in accordance with INCI as Cyclomethicone, amino-modified silicones (INCI: Amodimethicone) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Evonik.

Silicones which are particularly preferred in accordance with the invention are dimethicone and cyclomethicone.

Further suitable emollients are mono- and/or dialkyl carbonates of linear or branched C₆-C₂₂ fatty alcohols, such as Dicaprylyl Carbonate (Cetiol® CC) or dipropylheptyl carbonate (Cetiol® 4 All), Guerbet carbonates based on fatty alcohols having 6 to 18, preferably 8 to 10, carbon atoms, linear or branched, symmetrical unsymmetric dialkyl ethers having 6 to 22 carbon atoms per alkyl group, for example Dicaprylyl Ether (Cetiol® OE). Further suitable emollients are hydrocarbons such as mineral oils, Paraffinum Liquidum, Undecane/Tridecane (Cetiol® Ultimate), Hydrogenated Polyisobutene (Luvitol® Lite), substituted cyclohexanes, isoparaffins or paraffins.

Suitable Guerbet alcohols are those based on fatty alcohols having 6 to 18, preferably 8 to 10, carbon atoms (Eutanol® G, Eutanol® G 16).

More preferably, in the context of the present invention, the base mixture b1) comprises at least one emollient selected from the group formed by esters of C₂-C₁₂ dicarboxylic acids with linear or branched alcohols having 1 to 22 carbon atoms, mono- and/or dicarbonates of linear or branched C₆-C₂₂ fatty alcohols, hydrocarbons, esters of linear C₆-C₂₂ fatty acids with linear or branched C₆-C₂₂ fatty alcohols, liquid mono-/di-/triglyceride mixtures based on C₆-C₁₈ fatty acids, vegetable fats and oils, and Guerbet alcohols based on fatty alcohols having 6 to 18 carbon atoms.

In particular, the base mixture b1) comprises at least one emollient selected from the group formed by esters of C₄-C₈ dicarboxylic acids with linear or branched alcohols having 2 to 6 carbon atoms, mono- and/or dicarbonates of linear or branched C₆-C₁₂ fatty alcohols, hydrocarbons, esters of linear C₆-C₁₈ fatty acids with linear or branched C₆-C₂₂ fatty alcohols, liquid mono-/di-/triglyceride mixtures based on C₆-C₁₈ fatty acids, vegetable fats and oils, and Guerbet alcohols based on fatty alcohols having 8 to 10 carbon atoms.

In particular, recommended emollients in the base mixture are esters of linear and/or branched C₆-C₁₈ fatty acids with linear C₆-C₂₂ fatty alcohols, esters of dicarboxylic acids with linear or branched alcohols having 1 to 22 carbon atoms and/or linear and branched C₆-C₂₂ fatty alcohol carbonates and especially the adipic acid esters, sebacic esters or malic esters of linear or branched alcohols having 1 to 18 carbon atoms, esters of benzoic acid with linear and/or branched C₆-C₂₂ alcohols, esters of hydroxycarboxylic acids with linear C₆-C₂₂ fatty alcohols. Suitable examples are coco caprylate, lauryl lactate, C₁₂-C₁₅-alkyl benzoate, dibutyl adipate, dicaprylyl carbonate.

For cosmetic formulations such as skincare formulations, suitable emollients are esters of C₂-C₁₂ dicarboxylic acids with linear or branched alcohols having 1 to 22 carbon atoms, such as dibutyl adipate, mono- and/or dicarbonates of linear or branched C₆-C₂₂ fatty alcohols, such as dicapryl carbonate, hydrocarbons such as undecane/tridecane, esters of linear C₆-C₂₂ fatty acids with linear or branched C₆-C₂₂ fatty alcohols, such as ethylhexyl stearate, liquid mono-/di-/triglyceride mixtures based on C₆-C₁₈ fatty acids, such as Caprylic/Capric Triglyceride, vegetable fats and oils, such as shea butter, and Guerbet alcohols based on fatty alcohols having 8 to 18 carbon atoms, such as octyldodecanol.

Examples of particularly suitable emollients are dibutyl adipate, dioctyl carbonate, dipropylheptyl carbonate, octyldodecanol, undecane/tridecane, Caprylic/Capric Triglyceride, C₁₂-C₁₅-alkyl benzoate, ethylhexyl stearate, myristyl myristate, glyceryl oleate, isopropyl palmitate, dioctyl ether, octadecenyl docosenoate, rapeseed oil, wheatgerm oil, and Olus Oil.

Particularly suitable emollients are esters of dicarboxylic acids with linear or branched alcohols, for example dibutyl adipate, dioctyl carbonate and diisopropyl sebacate; triglycerides based on C₆-C₁₀ fatty acids; liquid mono-/di-/triglyceride mixtures based on C₆-C₁₈ fatty acids and esters of linear C₆-C₂₂ fatty acids with linear or branched C₆-C₂₂ fatty alcohols, for example myristyl myristate.

Interface-Active Substance

The base mixture in b1) also mandatorily comprises at least one interface-active substance. This may be nonionic, anionic, cationic and/or amphoteric or zwitterionic.

In one embodiment, recommended interface-active substances in the base mixture are at least one nonionic and/or anionic interface-active substance, especially for cosmetic formulations on the skin.

In a particular embodiment, recommended interface-active substances in the base mixture are at least one nonionic interface-active substance, especially for cosmetic formulations on the skin. In a further particular embodiment, recommended interface-active substances in the base mixture are at least one nonionic and one anionic interface-active substance, especially for cosmetic formulations on the skin.

Examples of suitable nonionic interface-active substances are
(1) Addition products of 2 to 50 mol of ethylene oxide and/or 1 to 20 mol of propylene oxide onto linear fatty alcohols having 8 to 40 carbon atoms, onto fatty acids having 12 to 40 carbon atoms and onto alkylphenols having 8 to 15 carbon atoms in the alkyl group (2) C₁₂-C₁₈ fatty acid mono- and diesters of addition products of 1 to 50 mol of ethylene oxide onto glycerol
(3) Sorbitan mono- and diesters of saturated and unsaturated fatty acids having 6 to 22 carbon atoms and the ethylene oxide addition products thereof
(4) Alkyl mono- and oligoglycosides, preferably having 8 to 22 carbon atoms in the alkyl radical, and ethoxylated analogs thereof
(5) Addition products of ethylene oxide onto castor oil and/or hydrogenated castor oil, for example with 2 to 15 mol or with 40 to 60 mol of ethylene oxide (6) Polyol and especially polyglyceryl esters, such as polyol poly-12-hydroxystearates, polyglyceryl polyhydroxystearate, polyglyceryl polyricinoleate, polyglyceryl diisostearate or polyglyceryl dimerate. Likewise suitable are mixtures of compounds of two or more of these substance classes
(7) Lecithins and phospholipids
(8) Partial esters based on linear, branched, unsaturated or saturated C₆-C₂₂ fatty acids, ricinoleic acid and 12-hydroxystearic acid and polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose), or mixed esters, for example glyceryl stearate citrate and glyceryl stearate lactate.
(9) Polysiloxane-polyalkyl-polyether copolymers and corresponding derivatives (called silicone emulsifiers)
(10) Mixed esters of pentaerythritol, fatty acids, citric acid and fatty alcohol and/or mixed esters of fatty acids having 6 to 22 carbon atoms, methylglucose and polyols, preferably glycerol or polyglycerol, and/or
(11) Mixed esters of fatty acids having 6 to 22 carbon atoms with sucrose

The addition products of ethylene oxide and/or of propylene oxide onto fatty alcohols, fatty acids, alkylphenols, glycerol mono- and diesters, and also sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. According to the degree of ethoxylation, the interface-active compounds can emulsify water in oil or oil in water. C_{12/18} fatty acid mono- and diesters of addition products of ethylene oxide onto glycerol are also known as refatting agents in cosmetic formulations.

Examples of suitable commercially available interface-active substances of the nonionic type are Cetyl Dimethicone Copolyol (e.g. Abil EM-90), Polyglyceryl-2 Dipolyhydroxystearate (e.g. Dehymuls PGPH), Polyglyceryl-3 Diisostearate (e.g. Lameform TGI), Polyglyceryl-4 Isostearate (e.g. Isolan GI 34), Polyglyceryl-3 Oleate (e.g. Isolan GO 33), Diisostearoyl Polyglyceryl-3 Diisostearate (e.g. Isolan PDI), Polyglyceryl-3 Methylglucose Distearate (e.g. Tego Care 450), Polyglyceryl-3 Beeswax (e.g. Cera Bellina), Polyglyceryl-4 Caprate (e.g. Polyglycerol Caprate T2010/90), Polyglyceryl-3 Cetyl Ether (e.g. Chimexane NL), Polyglyceryl-3 Distearate (e.g. Cremophor GS 32) and Polyglyceryl Polyricinoleate (e.g. Admul WOL 1403), Glyceryl Oleate (e.g. Monomuls 90-O 18), Alkyl Glucoside (e.g. Plantacare 1200, Emulgade PL 68/50, Montanov 68, Tego Care CG 90, Tego Glucosid L 55), Methyl Glucose Isostearate (e.g. Tego Care IS), Methyl Glucose Sesquistearate (Tego Care PS), Sucrose Ester (e.g. Crodesta F-10, F-20, F-50, F-70, F-110, F-160, SL-40, Emulgade® Sucro), ethoxylated and/or propoxylated fatty alcohols, fatty acids, castor oils and hydrogenated castor oils (e.g. Eumulgin® B1, B2, B3, BA 25, L, CO 40, CO 60, O 10, O 30, S 2, S 20, Cremophor® WO 7, Arlacel 989), PEG-30 Dipolyhydroxystearate (e.g. Arlacel P 135, Dehymuls LE), sorbitan esters, sorbitan esters ethoxylated and/or propoxylated, and mixtures thereof. A particularly effective mixture consists of Polyglyceryl-2 Dipolyhydroxystearate and Lauryl Glucoside and glycerol (e.g. Eumulgin VL 75). Also suitable are Polyglyceryl-4 Diisostearate/Polyhydroxystearate/Sebacate (Isolan GPS) or Diisostearoyl Polyglyceryl-3 Diisostearate (e.g. Isolan PDI).

Particularly advantageous nonionic interface-active substances are reaction products of poly-12-hydroxystearic acid with polyglycerols of the following homolog distribution (preferred amounts are given in brackets):
Glycerols: 5 to 35 (15 to 30) % by weight
Diglycerols: 15 to 40 (20 to 32) % by weight
Triglycerols: 10 to 35 (15 to 25) % by weight
Tetraglycerols: 5 to 20 (8 to 15) % by weight
Pentaglycerols: 2 to 10 (3 to 8) % by weight
Oligoglycerols: ad 100% by weight

In a preferred embodiment of the invention, the glyceryl ester used is the diester of polyhydroxystearic acid, Polyglyceryl-2 Dipolyhydroxystearate, which is sold, for example, by BASF Personal Care and Nutrition GmbH under the Dehymuls® PGPH name on its own, blends thereof under the Eumulgin® VL 75 name (blend with Lauryl Glucosides in a weight ratio of 1:1, O/W emulsifier) or Dehymuls® SBL name (W/O emulsifier) by BASF Personal Care and Nutrition Deutschland GmbH.

Nonionic interface-active compounds from the group of the alkyl mono- or alkyl oligoglycosides are particularly skin-friendly and can therefore be used either alone or in combination in the context of the invention. C₈-C₂₂-Alkyl mono- and oligoglycosides are known from the prior art. They are prepared especially by reacting glucose or oligosaccharides with primary alcohols having 8 to 22 carbon atoms, preferably 12 to 22, and more preferably 12 to 18 carbon atoms. As regards the glycoside radical, either monoglycosides, in which a cyclic sugar radical is glycosidically bonded to the fatty alcohol, or oligomeric glycosides with a degree of oligomerization up to preferably about 8 are suitable. The degree of oligomerization here is a statistical average value which is based on a homolog distribution customary for such technical-grade products. Products available under the Plantacare® name comprise a glucosidically bonded C₈-C₁₆-alkyl group onto an oligoglucoside radical having an average degree of oligomerization of 1 to 2. The acylglucamides derived from glucamine are also suitable.

Also suitable as nonionic interface-active compounds are substances such as lecithins and phospholipids. Examples of natural lecithins include the cephalins, which are also referred to as phosphatidic acids and are derivatives of 1,2-diacyl-sn-glycerol-3-phosphoric acids. In contrast, phospholipids are usually understood to mean mono- and preferably diesters of phosphoric acid with glycerol (glycerol phosphates), which are generally counted among the fats. In addition, sphingosines or sphingolipids are also possible.

Examples of nonionic interface-active compounds that may be present include silicone emulsifiers. These may be selected, for example, from the group of alkylmethicone copolyols and/or alkyldimethicone copolyols, especially from the group of compounds which are characterized by the following chemical structure:

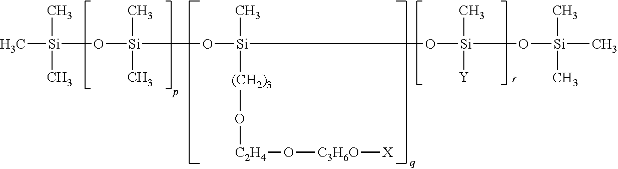

in which X and Y are independently selected from the group of H (hydrogen) and the branched and unbranched alkyl groups, acyl groups and alkoxy groups having 1-24 carbon atoms, p is 0-200, q is 1-40, and r is 1-100.

One example of silicone emulsifiers is that of dimethicone copolyols, which are sold by Evonik under the AXIL® B 8842, ABIL® B 8843, ABIL® B 8847, ABIL® B 8851, ABIL® B 8852, ABIL® B 8863, ABIL® B 8873 and ABIL® B 88183 trade names. A further example is Cetyl PEG/PPG-10/1 Dimethicone (Cetyl Dimethiconecopolyol), which is sold by Evonik under the ABIL® EM 90 brand name. A further example is Cyclomethicone Dimethiconecopolyol, which is sold by Evonik under the ABIL® EM 97 and ABIL® WE 09 brand name. Also suitable are Lauryl PEG/PPG-18/18 Methicone (Laurylmethiconecopolyol), available under the Dow Corning® 5200 Formulation Aid brand-name from the company Dow Corning Ltd., and Octyl Dimethicone Ethoxy Glucoside from Wacker.

Also suitable as interface-active compounds are zwitterionic, ampholytic and/or cationic compounds.

Interface-active compounds which bear at least one quaternary ammonium group and at least one —COO(−) or —SO₃(−) group in the molecule are referred to as zwitterionic.

Particularly suitable zwitterionic compounds are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazoline having in each case 8 to 18 carbon atoms in the alkyl or acyl group, and also cocoacylaminoethyl hydroxyethylcarboxymethylglycinate. A preferred zwitterionic interface-active compound is the fatty acid amide derivative known by the INCI name Cocamidopropyl Betaine.

Interface-active compounds which, aside from a C₈-C₁₈-alkyl or acyl group, comprise at least one free amino group and at least one —COOH or —SO₃H group in the molecule and are capable of forming internal salts are called ampholytic. Examples of suitable ampholytic compounds are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having about 8 to 18 carbon atoms in the alkyl group.

Usable cationic interface-active compounds are especially quaternary ammonium compounds. Preference is given to ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylbenzylammonium chloride and tricetylmethylammonium chloride. Additionally suitable are fatty amines such as Stearamidopropyl Dimethylamine (Dehyquart® S 18) and the very readily biodegradable quaternary ester compounds, for example the dialkylammonium methosulfates and methylhydroxyalkyldialkoyloxyalkylammonium methosulfates sold under the trade name Stepantex® and the corresponding products of the Dehyquart® series, be used as cationic interface-active compound. The term “ester quats” is generally understood to mean quaternized fatty acid triethanolamine ester salts. Likewise suitable are the quaternized protein hydrolyzates.

Also suitable are interface-active compounds having at least one anionic group such as a carboxylate, sulfate, sulfonate or phosphate group. Examples of suitable interface-active compounds, each in the form of their salts, are fatty acids, ether carboxylic acids, acyl sarcosides having 8 to 24 carbon atoms in the acyl group, acyl taurides having 8 to 24 carbon atoms in the acyl group, acyl isethionates having 8 to 24 carbon atoms in the acyl group, acyl glutamates having 8 to 24 carbon atoms in the acyl group, sulfosuccinic mono- and dialkyl esters having 8 to 24 carbon atoms in the alkyl group and sulfosuccinic monoalkyl polyoxyethyl esters having 8 to 24 carbon atoms in the alkyl group and 1 to 6 oxyethyl groups, linear alkanesulfonates having 8 to 24 carbon atoms, alkylarylsulfonates, linear alpha-olefinsulfonates having 8 to 24 carbon atoms, alpha-sulfo fatty acid methyl esters of fatty acids having 8 to 30 carbon atoms, alkyl sulfates, alkyl polyglycol ether sulfates, esters of tartaric acid and esters of citric acid, alkyl and/or alkenyl ether phosphates, sulfated fatty acid alkylene glycol esters, monoglyceride sulfates and monoglyceride ether sulfates and condensation products of C8-C30 fatty alcohols with protein hydrolyzates and/or amino acids and derivatives thereof, called protein fatty acid condensates, e.g. Lamepon®, Gluadin®, Hostapon® KCG or Amisoft.

Typically, the salts are selected from the sodium, potassium and ammonium salts, and the mono-, di- and trialkanolammonium salts having 2 to 4 carbon atoms in the alkanol group.

Suitable examples are sodium acylglutamate, Myristoyl Sarcosine, TEA-Lauroyl Sarcosinate, sodium lauroylsarcosinate and sodium cocoylsarcosinate, sodium or ammonium cocoylisethionate, dioctylsodium sulfosuccinate, disodium laureth sulfosuccinate, disodium laurylsulfosuccinate and disodium undecylenamido MEA sulfosuccinate, Dinatrium PEG-5 Laurylcitratsulfosuccinate, sodium, ammonium, magnesium, MIPA, TIPA laureth sulfate, sodium myrethsulfate and sodium C₁₂₁₃ parethsulfate, sodium C12-15 Pareth-15 Sulfonate, sodium, ammonium and TEA laurylsulfate, sodium lauroyltaurate and sodium methylcocoyltaurate, sodium laureth-13 carboxylate and Sodium PEG-6 Cocamide Carboxylate, Sodium PEG-7-Olive Oil Carboxylate, DEA-Oleth-10 Phosphate and Dilaureth-4 Phosphate, sodium cocomonoglyceridesulfate, Sodium C₁₂₋₁₄ Olefinsulfonate, sodium laurylsulfoacetate, Magnesium PEG-3 Cocamidsulfate, di-TEA-palmitoyl aspartate and Sodium Caprylic/Capric Glutamate, Palmitoyl Hydrolyzed Milk Protein, Sodium Cocoyl Hydrolyzed Soy Protein and Sodium/Potassium Cocoyl Hydrolyzed Collagen, calcium stearoyllactylate, Laureth-6 Citrate and Sodium PEG-4 Lauramide Carboxylate.

Anionic compounds of excellent suitability are alk(en)yl polyglycol ether citrates and especially mixtures of mono-, di- and triesters of citric acid and alkoxylated alcohols that conform to the formula (I):

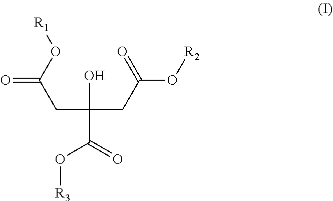

in which
R₁, R₂ and R₃ are independently hydrogen or the radical of the formula (II) R₄(OCH₂CHR₅)ₙ
in which
R₄ is a linear or branched alkyl and/or alkenyl radical having 6 to 22 carbon atoms,
R₅ is hydrogen or methyl radical and
n is a number from 1 to 20, with the condition that at least one of the R₁, R₂ and R₃ radicals is not hydrogen.

Typical examples of the alcohol moiety of the esters are addition products of an average of 1 to 20 mol, preferably 5 to 10 mol, of ethylene oxide and/or propylene oxide onto caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachidyl alcohol, gadoleyl alcohol, behenyl alcohol, erucyl alcohol and brassidyl alcohol, and technical grade mixtures thereof.

It is particularly preferable in the context of the invention to use, as anionic interface-active substances, salts of alkyl polyglycol ether sulfates, salts of acyl isethionates, salts of acyl glutamates, salts of mono- and dialkyl sulfosuccinates having 8 to 24 carbon atoms in the alkyl group, salts of alkyl sulfates, salts of alkyl phosphates, salts of fatty acids, citric esters of glyceryl stearate, and the alkyl polyalkylene glycol ether citrates that have already been discussed in detail above, especially the alkyl polyalkylene glycol ether citrates that have already been discussed in detail above.

In one embodiment of the present invention, for production of skin formulations, interface-active substances present as base mixture in b1) are nonionic and/or anionic interface-active compounds. Preferred nonionic interface-active compounds are those from the group formed by polyglyceryl esters, sucrose esters, ethoxylated fatty alcohols and ethoxylated fatty acids and alkyl (oligo)glucosides.

Preferred anionic interface-active compounds are those from the group formed by alkyl polyalkylene glycol ether citrates, salts of acyl glutamate, salts of mono- and dialkyl sulfosuccinates having 12 to 24 carbon atoms in the alkyl group, salts of alkyl sulfates and alkyl phosphates, and citric esters of Glyceryl Stearate and Sodium Lauryl Glucose Carboxylate.

A suitable subtype is that of mixtures consisting of two different nonionic interface-active compounds, preferably a mixture of 2 different interface-active compounds selected from the group formed by polyglyceryl esters, ethoxylated fatty alcohols and alkyl (oligo)glucosides.

A further suitable subtype is that of mixtures consisting of at least one anionic interface-active compound and at least one nonionic interface-active compound, preferably of one of the anionic interface-active compounds selected from the group of the alkyl polyalkylene glycol ether citrates, salts of acyl glutamate and salts of mono- and dialkyl sulfosuccinates having 12 to 24 carbon atoms in the alkyl group, and one of the nonionic interface-active compounds selected from the group of the polyglyceryl esters, sucrose esters and alkyl (oligo)glucosides.

In a further embodiment of the present invention, the interface-active substance present in the base mixture is one or more cationic and/or zwitterionic substances, preferably the cationic and/or zwitterionic compounds already mentioned above, preferably quaternary ammonium compounds, dialkylammonium methosulfates, methylhydroxyalkyldialkoyloxyalkylammonium methosulfates, quaternized fatty acid triethanolamine ester salts, fatty amines and betaines, especially Distearoyl Hydroxyethylmonium Methosulfate, Dicocoylethyl Hydroxyethylmonium Methosulfate, Hexadecyltrimethylammonium chloride and/or Stearamidopropyl Dimethylamine. This embodiment is recommended for production of cosmetic formulations for the hair.

Rheology Modifier

In the context of the invention, the base mixture in b1) mandatorily comprises a rheology modifier or no rheology modifier. Rheology modifiers may be present, for example, rather than in the base mixture, also in a formulation in a different dispensing vessel.

If present, the rheology modifiers are preferably selected from the group formed by
anionic, nonionic, cationic and/or zwitterionic polymers
inorganic substances selected from the group of the fumed silicas, bentonites and hectorites, where these may optionally be chemically modified, and
organic substances from the group of the fats, waxes, alcohols and/or hydrocarbons having a melting point above 45° C.

Polymers

Suitable rheology modifiers are anionic, nonionic, cationic and/or zwitterionic polymers having a natural or synthetic basis.

Examples of polymers having a natural basis are:
starch and modified starch
quaternized collagen polypeptides such as Lauryldimonium hydroxypropyl hydrolyzed collagen (Lamequat® L/Grunau)
polysaccharides, especially Xanthan Gum, Carrageenan, Gellan Gum, Acacia Gum, Tara Gum, Cellulose Gum, Scleroglucan, Glucomannan, Guar gum, Agar-Agar, Alginate, Pectin,
Chemically modified polysaccharides such as methyl hydroxyethylcellulose, hydroxyethyl- and hydroxypropylcellulose, carboxymethylcellulose
modified guar polymers, such as the Jaguar® brands from Solvay
derivatized cellulose ethers.

Examples of anionic polymers suitable as rheology modifier are polymers or copolymers comprising carboxylic acid groups in particular. Suitable carboxylic acid-group-containing polymers are obtainable, for example, by free-radical polymerization of α, β-ethylenically unsaturated monomers m1). Monomers used here comprise at least one free-radically polymerizable, α, β-ethylenically unsaturated double bond and at least one anionogenic and/or anionic group per molecule. Suitable monomers are monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers additionally include the monoesters of monoethylenically unsaturated dicarboxylic acids having 4 to 10, preferably 4 to 6, carbon atoms, for example of maleic acid, such as monomethyl maleate. The monomers also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloyloxypropylsulfonic acid, 2-hydroxy-3-methacryloyloxypropylsulfonic acid, styrenesulfonic acid, 1-acrylamido-2-methylpropanesulfonic acid, vinylphosphonic acid and allylphosphonic acid. The monomers also include the salts of the abovementioned acids, in particular the sodium, potassium and ammonium salts, and the salts with the abovementioned amines. The monomers can be used as they are or as mixtures with one another.

The monomer is preferably selected from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, more preferably acrylic acid, methacrylic acid and mixtures thereof. Preference is given to copolymers of acrylic acid and/or methacrylic acid that have molecular weight in the range from 10 000 to 10 000 000 daltons.

Additionally preferred are copolymers of the abovementioned monomers with monomers m2. Suitable monomers m2) are methyl (meth)acrylate, methyl ethacrylate, ethyl (meth)acrylate, ethyl acrylate, tert-butyl (meth)acrylate, tert-butyl ethacrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arachinyl (meth)acrylate, behenyl (meth)acrylate, lignocerenyl (meth)acrylate, cerotinyl (meth)acrylate, melissinyl (meth)acrylate, palmitoleyl (meth)acrylate, oleyl (meth)acrylate, linoleyl (meth)acrylate, linolenyl (meth)acrylate, stearyl (meth)acrylate, lauryl (meth)acrylate and mixtures thereof.

Suitable monomers m2) are additionally acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide, N-(n-octyl)(meth)acrylamide, N-(1,1,3,3-tetramethylbutyl)(meth)acrylamide, N-ethylhexyl(meth)acylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acylamide, N-palmityl(meth)acrylamide, N-heptadecyl(meth)acrylamide, N-nonadecyl(meth)acylamide, N-arachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleyl(meth)acrylamide, N-oleyl(meth)acrylamide, N-linoleyl(meth)acrylamide, N-linolenyl(meth)acrylamide, N-stearyl(meth)acrylamide and N-lauryl(meth)acrylamide.

Anionic polymers preferred as carboxylic acid group-containing polymers are, for example, homopolymers and copolymers of acrylic acid and methacrylic acid and salts thereof. These also include crosslinked polymers of acrylic acid, as are available under the INCI name Carbomer. Such crosslinked homopolymers of acrylic acid are commercially available, for example, under the Carbopoll® name from Lubrizol. Preference is also given to hydrophobic modified crosslinked and uncrosslinked polyacrylic copolymers, such as Carbopol® Ultrez 21 from Lubrizol.

Further examples of suitable anionic polymers are copolymers of acrylic acid and acrylamide and salts thereof; sodium salts of polyhydroxycarboxylic acids, polyurethanes and polyureas. Particularly suitable polymers are copolymers of (meth)acrylic acid and polyether acrylates, where the polyether chain is terminated with a C8-C30-alkyl radical. These include, for example, Acrylates/Beheneth-25-Methacrylate copolymers which are available under the Aculyn® name from Dow. Suitable polymers are additionally copolymers of acrylic acid or methacrylic acid with hydrophobic monomers, for example C4-C30-alkyl esters of meth(acrylic acid), C4-C30-alkyl vinyl esters, C4-C30-alkyl vinyl ethers and hyaluronic acid.

Likewise suitable as rheology modifiers are cationic polymers, for example what are called quaternary ammonium compounds with the INCI name Polyquaternium-22, Polyquaternium-37, Polyquaternium-39, Polyquaternium-47 or Polyquaternium-86.

In addition, it is also possible to use nonionic polymers as rheology modifiers, such as polyethylene glycols having an average molar mass of >800 g/mol or hydrophobically modified, ethoxylated urethane polymers (HEUR for short) such as Polyurethane-39.

Inorganic Substances

In addition, inorganic substances that can also function as rheology modifiers are those selected from the group of the fumed silicas, bentonites and hectorites, for example hydrophilic and hydrophobic fumed silicas that are sold under the Aerosil® trade name inter alia, and bentonites and hectorites or hydrophobically modified hectorite and bentonites such as Benzyldimethylstearylammonium Hectorite, Dimethyldioctylammonium Hectorite or Quaternium-18 Hectorite, and also Benzyldimethylstearylammonium Bentonite or Quaternium-18 Bentonite, which are available, for example, under the Bentone®, Claytone®, Tixogel® trade names.

Organic Substances

In addition, organic substances that can also function as rheology modifiers are those selected from the group of the fats, waxes, alcohols and or hydrocarbons having a melting point above 45° C. Rheology modifiers may also be wholly or partly wax esters, i.e. compounds having a melting point of 45° C., such as mono-, di- and/or triglycerides, such as the Cutina® MD or Cutina® GMS (glyceryl stearate) products marketed by BASF Personal Care and Nutrition Deutschland GmbH & Co. KG. Usable rheology modifiers are, for example, also natural vegetable waxes such as candelilla wax, carnauba wax, japan wax, rice germ oil wax, sugarcane wax, montan wax, sunflower wax, and animal waxes such as beeswax.

It is also possible to use hydrogenated or hardened waxes such as the mineral waxes, for example ceresin and ozokerite, or the petrochemical waxes, such as petrolatum, paraffin waxes and microwaxes. Usable rheology modifiers also include chemically modified waxes such as montan ester waxes, Sasol waxes and hydrogenated jojoba waxes. Synthetic waxes usable in accordance with the invention include, for example, wax-like polyalkylene waxes and polyethylene glycol waxes.

Further usable waxes are esters of aromatic carboxylic acids, dicarboxylic acids, tricarboxylic acids and hydroxycarboxylic acids (e.g. 12-hydroxystearic acid) and saturated and/or unsaturated, branched and/or unbranched alcohols. Examples of such esters are the C₁₆-C₄₀-alkyl stearates, C₂₀-C₄₀-alkyl stearates (e.g. Kesterwachs K82H), C₂₀-C₄₀-dialkyl esters of dimer acids, C₁₈-C₃₈-alkylhydroxystearoyl stearates or C₂₀-C₄₀-alkyl erucates. Also suitable are C₃₀-C₅₀-alkyl beeswax, tristearyl citrate, triisostearyl citrate, trilauryl citrate, ethylene glycol dipalmitate, ethylene glycol distearate, ethylene glycol di(12-hydroxystearate), stearyl stearate, palmityl stearate, stearyl behenate, cetearyl behenate, glyceryl mono/dilaurate, -palmitate, -myristate or -stearate, triesters of glycerol with a hydroxystearic acid (Cutina® HR) or glyceryl tristearate, glyceryl tribehenate (e.g. Syncrowax® HRC), glyceryl tripalmitate, or the triglyceride mixtures known by the Syncrowax® HGLC name.

Rheology modifiers present may also be fatty alcohols, fatty acids (hydrolysed) and/or glyceryl mono-, di- and/or tri-fatty acid esters and wax esters. Typical examples are cetyl alcohol (Lanette® 16), palmoleyl alcohol, stearyl alcohol (Lanette® 18), cetearyl alcohol (Lanette® O), isostearyl alcohol, oleyl alcohol, elaidyl alcohol, petroselyl alcohol, linolyl alcohol, linolenyl alcohol, elaeostearyl alcohol, arachyl alcohol, gadoleyl alcohol, behenyl alcohol (Lanette® 22), erucyl alcohol and brassidyl alcohol and technical grade mixtures thereof, which are obtained, for example, in the high pressure hydrogenation of technical grade methyl esters based on fats and oils or aldehydes from Roelen's oxo synthesis and also as monomer fraction in the dimerization of unsaturated fatty alcohols. Particularly preferred fatty alcohols are cetyl alcohol, stearyl alcohol and cetearyl alcohol. Examples of glyceryl esters are glyceryl laurate, such as Monomuls® 90-L-12, glyceryl oleate, such as Monomuls® 90-O 18, glyceryl stearate, such as Cutina® GMS V and Cutina® GMS V/MB, glycol distearate, such as Cutina® AGS, sorbitan stearate, such as Dehymuls® SMS, Cutina® HVG.

Particularly preferred rheology modifiers from the group of the polymers are anionic, nonionic or cationic, synthetic and/or natural polymers, especially selected from the group formed by polyacrylic acids and alkali metal salts thereof, copolymers of polyacrylic acid and alkali metal salts thereof, copolymers of (meth)acrylic acid and polyether acrylates, where the polyether chain is terminated by a C8-C30-alkyl radical, copolymers of acrylamidomethylpropanesulfonic acid and alkali metal salts thereof, and the polysaccharides.

From the group of the organic substances, rheology modifiers are more preferably fatty alcohols, glyceryl mono-, di- and/or tri-fatty acid esters, and wax esters.

In a particularly preferred embodiment, the base mixture in b1) comprises a combination of various rheology modifiers where one of the rheology modifiers is selected from the above-defined group of inorganic substances formed by the optionally chemically modified hectorites and bentonites. Such a combination can improve the thermal stability and long-term stability both of the base mixture and of the cosmetic formulation produced.

Preferred combinations of the rheology modifiers are mixtures of the hectorites and/or bentonites that have optionally been hydrophobically modified with anionic, nonionic or cationic, synthetic and/or natural polymers or mixtures of the hectorites and/or bentonites that have optionally been hydrophobically modified with organic substances selected from the group of the fatty alcohols, glyceryl mono-, di- and/or tri-fatty acid esters, and wax esters.

Particularly preferred combinations of the rheology modifiers are mixtures of the hectorites and/or bentonites that have optionally been hydrophobically modified with polymers selected from the group of polyacrylic acids and alkali metal salts thereof, copolymers of polyacrylic acid and alkali metal salts thereof, copolymers of (meth)acrylic acid and polyether acrylates, where the polyether chain is terminated by a C8-C30-alkyl radical, copolymers of acrylamidomethylpropanesulfonic acid and alkali metal salts thereof and the polysaccharides, and mixtures of the hectorites and/or bentonites that have optionally been hydrophobically modified with organic substances selected from the group of the fatty alcohols, glyceryl mono-, di- and/or tri-fatty acid esters, and wax esters.

More preferably, rheology modifiers are selected from the group of the natural or synthetic-based anionic, nonionic, cationic and/or zwitterionic polymers, inorganic substances selected from the group of fumed silicas, bentonites and hectorites, and organic substances from the group of fats, waxes, alcohols and/or hydrocarbons having a melting point above 45° C., fatty alcohols, polysaccharides, especially selected from the group of fatty alcohols and polysaccharides.

UV Light Protection Filters (Also Called UV Filters)

Suitable UV light protection filters organic substances that are liquid at room temperature or crystalline (light protection filters) which are capable of absorbing ultraviolet rays and releasing the energy absorbed again in the form of longer-wave radiation, for example heat. UV filters may be oil-soluble or water-soluble. Examples of typical oil-soluble UV-B filters or broad-spectrum UV-A/B filters include:
3-benzylidenecamphor or 3-benzylidenenorcamphor (Mexoryl SDS 20) and derivatives thereof, e.g. 3-(4-methylbenzylidene)camphor, as described in EP 0693471 B1;
3-(4′-trimethylammonium)benzylidenebornan-2-one methylsulfate (Mexoryl SO);
polymer of N-{(2 and 4)-[2-oxoborn-3-ylidene)methyl}benzyl]acrylamide (Mexoryl SW);
2-(2H-benzotriazol-2-yl)-4-methyl-6-(2-methyl-3-(1,3,3,3-tetramethyl-1-(trimethylsilyloxy)disiloxanyl)propyl)phenol (Mexoryl XL);
4-aminobenzoic acid derivatives, preferably 2-ethylhexyl 4-(dimethylamino)benzoate, 2-octyl 4-(dimethylamino)benzoate and amyl 4-(dimethylamino)benzoate (Uvinul A Plus);
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, for example 2,4,6-trianilino(p-carbo-2′-ethyl-1′-hexyloxy)-1,3,5-triazine and 2,4,6-tris[p-(2-ethylhexyloxycarbonyl)anilino]-1,3,5-triazine (Uvinul T 150), as described in EP 0818450 A1, or bis(2-ethylhexyl) 4,4′-[(6-[4-((1,1-dimethylethyl)aminocarbonyl)phenylamino]-1,3,5-triazine-2,4-diyl)diimino]benzoate (Uvasorb® HEB);
2,2-(methylenebis(6-(2H-benzotriazol-2-yl)-4-(1,1,3,3-tetramethylbutyl)phenol) (Tinosorb M);
2,4-bis[4-(2-ethylhexyloxy)-2-hydroxyphenyl]-6-(4-methoxyphenyl)-1,3,5-triazine (Tinosorb S);
propane-1,3-diones, for example 1-(4-tert-butylphenyl)-3-(4′-methoxyphenyl)propane-1,3-dione;
ketotricyclo(5.2.1.0)decane derivatives, as described in EP 0694521 B1;
dimethicodiethyl benzalmalonates (Parsol SLX).

Useful water-soluble UV filters include:
2-phenylbenzimidazole-5-sulfonic acid and the alkali metal, alkaline earth metal, ammonium, alkylammonium, alkanolammonium and glucammonium salts thereof;
2,2-(1,4-phenylene)bis(1H-benzimidazole-4,6-disulfonic acid, monosodium salt) (Neo Heliopan AP);
sulfonic acid derivatives of benzophenones, preferably 2-hydroxy-4-methoxybenzophenone-5-sulfonic acid and its salts;
3,3′-(1,4-phenylenedimethine)bis(7,7-dimethyl-2-oxobicyclo[2.2.1]heptane-1-methanesulfonic acid) and salts (Mexoryl SX);
3-(4′-sulfo)benzylidenebornan-2-one and salts (Mexoryl SL);
sulfonic acid derivatives of 3-benzylidenecamphor, for example 4-(2-oxo-3-bornylidenemethyl)benzenesulfonic acid and 2-methyl-5-(2-oxo-3-bornylidene)sulfonic acid and salts thereof.

Useful organic particulate UV filters include:
Methylene Bis-Benzotriazolyl Tetramethylbutylphenol;
Tris-Biphenyl triazine
Phenylene bis-diphenyltriazine;
Bis-(Diethylaminohydroxybenzoyl Benzoyl) Piperazine.

In one embodiment, preference is given to using water-dispersed UV filters, for example Methylene Bis-Benzotriazolyl Tetramethylbutylphenol, Tris-Biphenyl triazine, Phenylene bis-diphenyltriazine, Bis-(Diethylaminohydroxybenzoyl Benzoyl) Piperazine and Bis-Ethylhexyloxyphenol Methoxyphenyl Triazine. Particular preference is given to using water-dispersed UV filters selected from the group consisting of Methylene Bis-Benzotriazolyl Tetramethylbutylphenol, Tris-Biphenyl triazine and Bis-Ethylhexyloxyphenol Methoxyphenyl Triazine.

Useful typical UVA filters are especially derivatives of benzoylmethane, for example 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF), and also benzoic acid 2-[4-(diethylamino)-2-hydroxy-benzoyl]hexyl ester (Uvinul® A plus).

The UVA and UVB filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Combinations of this type are advantageously combined with water-soluble filters, for example 2-phenylbenzimidazole-5-sulfonic acid and the alkali metal, alkaline earth metal, ammonium, alkylammonium, alkanolammonium and glucammonium salts thereof.

Suitable UV light protection filters are especially the substances approved according to Annex VII of the Commission Directive (in the version: Commission Directive 2005/9/EC of Jan. 28, 2005 amending Council Directive 76/768/EEC, concerning cosmetic products, for the purposes of adapting Annexes VII thereof to technical progress), to which reference is explicitly made here. Very particularly suitable UV light protection filters are the substances that are approved according to Annex VI of Regulation (EC) No 1223/2009 of the European Parliament and of the Council on cosmetic products, to which reference is hereby made explicitly.

The base mixture in b1) preferably comprises at least one triazine derivative UV filter, preferably selected from the group consisting of tris(2-ethylhexyl) 4,4′,4″-(1,3,5-triazine-2,4,6-triyltriimino)tribenzoate (INCI: Ethylhexyl Triazone; Uvinul T 150); bis(2-ethylhexyl) 4,4′-{[6-({4-[(2-methyl-2-propanyl)carbamoyl]phenyl}amino)-1,3,5-triazine-2,4-diyl]diimino}dibenzoate (INCI: Diethylhexyl butamido triazone; Uvasorb HEB); 2,4-bis{[4-(2-ethylhexyloxy)-2-hydroxy]phenyl}-6-(4-methoxyphenyl)-1,3,5-triazine (INCI: bis-ethylhexyloxyphenol methoxyphenyl triazine; Tinosorb S), and mixtures thereof, and at least one UV-A filter, preferably selected from the group consisting of Diethylamino Hydroxybenzoyl Hexyl Benzoate (DHHB), Butylmethoxydibenzoylmethane (BMDBM), and mixtures thereof.

In addition to the soluble substances mentioned, insoluble light protection pigments, specifically finely dispersed metal oxides and salts, are also useful for this purpose. Examples of suitable metal oxides are especially zinc oxide and titanium dioxide, and additionally oxides of iron, of zirconium, of silicon, of manganese, of aluminum and of cerium, and mixtures thereof. The salts used may be silicates (talc), barium sulfate or zinc stearate. The oxides and salts are used in the form of the pigments for skincare and skin-protecting emulsions, and also for decorative cosmetics. The particles should have a mean diameter of less than 100 nm, preferably between 5 and 50 nm and especially between 15 and 30 nm. They may have a spherical shape, but it is also possible to use those particles which have an ellipsoidal shape or a shape which deviates in some other way from the spherical configuration. The pigments may also be in surface-treated form, i.e. hydrophilized or hydrophobized. Typical examples are coated titanium dioxides, for example T 805 titanium dioxide (Degussa) or Eusolex® T, Eusolex® T-2000, Eusolex® T-Aqua, Eusolex® AVO, Eusolex® T-ECO, Eusolex® T-OLEO and Eusolex® T-S(Merck). Typical examples are zinc oxides, for example Zinc Oxide neutral, Zinc Oxide NDM (Symrise) or Z-Cote® (BASF) or SUNZnO-AS and SUNZnO-NAS (Sunjun Chemical Co. Ltd.). Suitable hydrophobic coating agents are in particular silicones and specifically trialkoxyoctylsilanes or simethicones. In sunscreen compositions, preference is given to using so-called micropigments or nanopigments. Preference is given to using micronized zinc oxide. Further suitable UV light protection filters can be found in the review by P. Finkel in SOFW-Journal 122, 8/1996, pages 543-548 and Parf. Kosm. Volume 80, no. 3/1999, pages 10 to 16.

These UV light protection filters are commercially available, for example, under the following trade names:

NeoHeliopan® MBC (INCI: 4-Methylbenzylidene Camphor; manufacturer: Symrise); NeoHeliopan® BB (INCI: Benzophenone-3, manufacturer: Symrise); Parsol®1789 (INCI: Butyl Methoxydibenzoylmethane, manufacturer: Hoffmann-La Roche (Givaudan); Tinosorb® S (INCI: Bis-Ethylhexyloxyphenol Methoxyphenyl Triazine); Tinosorb® M (INCI: Methylene Bis-Benzotriazolyl Tetramethylbutylphenol): manufacturer: BASF; Uvasorb® HEB (INCI: Diethylhexyl Butamido Triazone, manufacturer: 3V Inc.), Uvinul® T 150 (INCI: Ethylhexyl Triazone, manufacturer: BASF AG); Uvinul® A plus (INCI: Diethylamino Hydroxybenzoyl Hexyl Benzoate: Manufacturer: BASF AG; Mexoryl® SO: 3-(4′-trimethylammonium)benzylidenebornan-2-one methylsulfate INCI: Camphor Benzalkonium Methosulfate; Mexoryl® SX: 3,3′-(1,4-phenylenedimethine)bis(7,7-dimethyl-2-oxobicyclo[2.2.1]heptane-1-methanesulfonic acid), CTFA: INCI Terephthalylidene Dicamphor Sulfonic Acid; Mexory® SL: 3-(4′-sulfo)benzylidenebornan-2-one, INCI Benzylidene Camphor Sulfonic Acid; Mexoryl® SW: polymers of N-{(2 and 4)-[2-oxoborn-3-ylidene)methyl}benzyl]acrylamid, INCI Polyacrylamidomethyl Benzylidene Camphor; Mexoryl® SL: 2-(2H-benzotriazol-2-yl)-4-methyl-6-(2-methyl-3-(1,3,3,3-tetramethyl-1-(trimethylsilyloxy)disiloxanyl)propyl)phenol; INCI: DROMETRIZOLE TRISILOXANE; Parsol® SLX: Dimethicodiethylbenzalmalonate, INCI Polysilicone-15.

Further examples are pigmentary light filters, for example inorganic pigmentary light filters, such as titanium dioxide and zinc oxide and/or organic pigmentary light filters such as methylenebisbenzotriazolyltetramethylbutylphenol (Tinosorb M).

Particular preference is given to using organic UV filters.

Amounts in the Base Mixture in b1) In the context of the present invention, the base mixture in b1) comprises at least one emollient, at least one interface-active substance, at least one UV filter and at least one rheology modifier or no rheology modifier, where the weight ratio of emollient:interface-active substance is preferably in the range from 1:4 to 25:1, more preferably in the range from 1:2 to 25:1. In one embodiment of the invention, the base mixture for the production of skin formulations in b1) preferably comprises a weight ratio of emollient:interface-active substance in the range from 1:1 to 25:1, more preferably 1.5:1 to 12:1, more preferably 2:1 to 10:1 and especially 3:1 to 8:1.

It is especially suitable when the base mixture in b1) has a weight ratio of emollient:interface-active substance+rheology modifier in the range from 1:10 to 10:1. In one embodiment of the invention, the base mixture in b1) for the production of skin formulations preferably comprises a weight ratio of emollient:interface-active substance+rheology modifier in the range from 1:2 to 8:1, more preferably in the range from 1:1 to 7:1 and especially in the range from 2:1 to 7:1.

It is especially suitable when there is a weight ratio of emollient:UV filter in the base mixture in b1) in the range from 1:5 to 10:1, more preferably 1:2 to 8:1, even more preferably 1:1.5 to 6:1 and especially 1:1 to 4:1.

It is especially suitable when there is a weight ratio of UV filter:interface-active substance in the base mixture in b1) in the range from 1:5 to 10:1, more preferably 1:2 to 8:1, even more preferably 1:1 to 6:1 and especially 1.5:1 to 5:1.

Further Cosmetic Ingredients

The base mixture in b1) may comprise further cosmetic ingredients. Cosmetic ingredients in the context of the invention may be cosmetic auxiliaries and additives or else active cosmetic ingredients.

The base mixture in b1) preferably comprises the auxiliaries and additives that are customarily used in cosmetics, such as water, stabilizers, solvents, solubilizers, preservatives, neutralizing agents and buffers, complexing agents and the like.

Cosmetic Auxiliaries and Additives

Neutralizing Agents and Buffers

Examples of suitable neutralizing agents are the compatible acids or bases known in the cosmetics industry, which are likewise listed in the Cosmetics Directive. Buffers ensure the pH stability of the cosmetic formulations. Primarily citrate, lactate and phosphate buffers are used.

Preservative

Suitable preservatives are, for example, ethanol, isopropanol, phenoxyethanol, the combination of phenoxyethanol with methyldibromoglutaronitrile, formaldehyde solution, parabens, pentanediol or sorbic acid, benzoic acid and salts thereof, Benzyl Alcohol, Benzyl Salicylate, urea condensates, p-hydroxybenzoic esters, Dehydroacetic Acid, methylthiazolinone or sorbic acid and salts thereof, and also the silver complexes known by the Surfacine® name, and the further substance classes listed in Annex 6, parts A and B, of the Cosmetics Directive. Additionally used are substances which function as preservation aids such as Ethylhexylglycerin and Caprylyl Glycol, and polyols or alcohols such as isopropyl alcohol, propanediol, phenylpropanol, phenethyl alcohol and undecyl alcohol and also the silver complexes known by the Surfacine® name. Additionally suitable as preservatives are the 1,2-alkanediols having 5 to 8 carbon atoms, which are described in WO07/048757.

Advantageous preservatives in the context of the present invention are, for example, formaldehyde releasers (for example DMDM Hydantoin, commercially available, for example, under the Glydant® trade name (Lonza)), iodopropyl butylcarbamates (e.g. Glycacil-L®, Glycacil-S® (Lonza), Dekaben® LMB (Jan Dekker)), parabens (alkyl p-hydroxybenzoate, for example methyl-, ethyl-, propyl- and/or butylparaben), Dehydroacetic Acid (Euxyl® K 702 (Schülke&Mayr), phenoxyethanol, ethanol, benzoic acid. It is also advantageous to use what are called preservation aids, for example octoxyglycerin, glycine, soya etc.

The table below gives an overview of possible preservatives that are suitable.

| | |
|---|---|
| E200 | Sorbic acid |
| E201 | Sodium sorbate |
| E202 | Potassium sorbate |
| E203 | Calcium sorbate |
| E210 | Benzoic acid |
| E211 | Sodium benzoate |
| E212 | Potassium benzoate |
| E213 | Calcium benzoate |
| E214 | Ethyl p-hydroxybenzoate |
| E215 | Ethyl p-hydroxybenzoate Na salt |
| E216 | n-Propyl p-hydroxybenzoate |
| E217 | n-Propyl p-hydroxybenzoate Na salt |
| E218 | Methyl p-hydroxybenzoate |
| E219 | Methyl p-hydroxybenzoate Na salt |
| E220 | Sulfur dioxide |
| E221 | Sodium sulfite |
| E222 | Sodium hydrogensulfite |
| E223 | Sodium disulfite |
| E224 | Potassium disulfite |
| E226 | Calcium sulfite |
| E227 | Calcium hydrogensulfite |
| E228 | Potassium hydrogensulfite |
| E230 | Biphenyl (diphenyl) |
| E231 | Orthophenylphenol |
| E232 | Sodium orthophenylphenoxide |
| E233 | Thiabendazole |
| E235 | Natamycin |
| E236 | Formic acid |
| E237 | Sodium formate |
| E238 | Calcium formate |
| E239 | Hexamethylenetetramine |
| E249 | Potassium nitrite |
| E250 | Sodium nitrite |
| E251 | Sodium nitrate |
| E2li | Potassium nitrate |
| E280 | Propionic acid |
| E281 | Sodium propionate |
| E282 | Calcium propionate |
| E283 | Potassium propionate |
| E290 | Carbon dioxide |

Also advantageous are preservatives or preservation aids that are commonly used in cosmetics, such as dibromodicyanobutane (2-bromo-2-bromomethylglutaronitrile), phenoxyethanol, 3-iodo-2-propynylbutyl carbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, salicylic acid and salicylates.

Particularly preferred preservatives are sodium benzoate, parabens (methyl-, ethyl-, propyl- and/or butylparaben) and/or phenoxyethanol.

Complexing Agents

Suitable complexing agents are salts of ethylenediaminetetraacetic acid, of nitrilotriacetic acid, of iminodisuccinic acid, or phosphates.

Stabilizers

Stabilizers used may be metal salts of fatty acids, for example magnesium stearate/ricinoleate, aluminum stearate/ricinoleate and/or zinc stearate/ricinoleate, in order to improve emulsion stability, but also compounds such as tris(tetramethylhydroxypiperidinol) citrates as light stabilizers in order to prevent discoloration or changes in odor in the formulations.

Solubilizers

Solubilizers used may, for example, be ethylene oxide adducts having an ethoxylation level of 20 to 60 onto castor oil or hydrogenated castor oil, ethylene oxide adducts and/or propylene oxide adducts having 2-20 ethylene oxide units and 1 to 20 mol of propylene oxide units onto fatty alcohols having 8 to 40 carbon atoms or onto fatty acids having 12 to 40 carbon atoms or onto alkylphenols having 8 to 15 carbon atoms in the alkyl group. Also suitable are ethylene oxide adducts of 1 to 50 mol of ethylene oxide units onto C12-C18 fatty acid mono- and diesters of glycerol or polyglycerol or sorbitan. Preferentially suitable solubilizers are Eumulgin® HRE 40 (INCI: PEG-40 Hydrogenated Castor Oil), Eumulgin® HRE 60 (INCI: PEG-60 Hydrogenated Castor Oil), Eumulgin® L (INCI: PPG-1-PEG-9 Lauryl Glycol Ether), and Eumulgin® SML 20 (INCI: Polysorbate-20).

Solvents

Suitable solvents are alcohols such as ethanol, propanediol or glycerol.

Water

In the context of the present invention, water may likewise be present as ingredient in the base mixture in b1). In the present description, water is explicitly excluded and is not covered by the term “solvent”. In addition, the amount of water is explicitly stated in the present description.

Active Cosmetic Ingredients

The base mixture in b1) may already also comprise active cosmetic ingredients that are utilized for the personalization of the cosmetic formulations, for example active biogenic ingredients, secondary light stabilizers, self-tanning agents, insect repellents, antioxidants, film formers, sensory additives, effect pigments, tyrosine inhibitors (depigmenting agents), coolants, perfume oils, dyes and humectants. This is advantageous, for example, when, according to the demands of the user or desired effect, extremely high amounts of active cosmetic ingredients are to be present in a cosmetic formulation.

It is also possible to use secondary light stabilizers of the antioxidant type which interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, #-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to mol/kg), also (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids), suitable in accordance with the invention, of these specified active ingredients.

Self-Tanning Agents

Self-tanning agents are understood to mean substances which cause browning of the skin. Examples include dihydroxyacetone, erythrulose and alpha, beta-unsaturated aldehydes, which react with the amino acids in the skin in the manner of a Maillard reaction to give colored compounds. Useful further active ingredients for self-tanning agents include natural or synthetic ketols or aldols. Examples of suitable active ingredients include dihydroxyacetone, erythrulose, glycerolaldehyde, alloxane, hydroxymethylglyoxal, gamma-dialdehyde, 6-aldo-D-fructose, ninhydrin and meso-tartaraldehyde. Suitable self-tanning agents are especially dihydroxyacetone and/or erythrulose.

Active Biogenic Ingredients

Active biogenic ingredients present may be active ingredients having a natural basis, preferably those that improve skin properties, for example tocopherol, tocopherol acetate, tocopherol palmitate, ascorbic acid, (deoxy)ribonucleic acid and the fragmentation products thereof, β-glucans, retinol, bisabolol, allantoin, phytantriol, panthenol, AHA acids, amino acids, ceramides, pseudoceramides, essential oils, plant extracts, for example aloe vera, prunus extract, bambara nut extract and vitamin complexes.

The following active biogenic ingredients are suitable as humectants for the skin: AMC® Advanced Moisture Complex NP (INCI: Glycerin (and) Water (and) Sodium PCA (and) Urea (and) Trehalose (and) Hexylene glycol (and) Polyquaternium-51 (and) Triacetin (and) Caprylyl Glycol (and) Sodium Hyaluronate); Hyalurosmooth® LS8998 (INCI: Water (and) Cassia angustifolia Seed Polysaccharide); Hyalurosmooth® LS 8997 (INCI: Cassia angustifolia Seed Polysaccharide); Irwinol® (INCI: Octyldodecanol (and) Irvingia Gabonensis Kernel Butter (and) Hydrogenated Coco-Glycerides); Lipofructyl® Argan (INCI: Argania Spinosa Kernel Oil); Melhydran® (INCI: Water (and) Butylene Glycol (and) Honey Extract (and) Glycerin (and) Urea (and) Sodium Lactate (and) Arginine HCl (and) Lysine HCl (and) Ornithine HCl); Oligolin® (INCI: Hydrolyzed Linseed Extract); PatcH₂O® (INCI: Water (and) Glycerin (and) Trehalose (and) Urea (and) Serine (and) Pentylene Glycol (and) Glyceryl Polyacrylate (and), Algin (and), Caprylyl Glycol (and) Sodium Hyaluronate (and), Pullulan (and) Disodium Phosphate (and) Potassium Phosphate); Relipidium® (INCI: Hydrolyzed Yeast Protein (and) Butylene Glycol (and) Pentylene Glycol); Sphingoceryl® VEG (INCI: Octyldodecanol (and) Hydrogenated Coco-Glycerides (and) Helianthus Annuus (Sunflower) Seed Extract).

The following active biogenic ingredients are suitable as anti-aging agents: AH-Care® (INCI: Water (and) Lactic Acid (and) Arginine); Argassential® (INCI: Argania Spinosa Fruit Extract (and) Dicaprylyl Ether (and) Sorbitol (and) Polyglyceryl-2 Dipolyhydroxystearate (and) Lauryl Glucoside (and) Glycerin (and) Water); Argatensyl® (INCI: Argania Spinosa Kernel Extract (and) Sodium Cocoyl Glutamate); Collalift® 18 (INCI: Glycerin (and) Water (and) Khaya Senegalensis Bark Extract (and) Maltodextrin); Deliner® (INCI: Water (and) Butylene Glycol (and) Pentylene Glycol (and) Zea mays (Corn) Kernel Extract (and) Xanthan Gum); Dermagenist® (INCI: Maltodextrin (and) Origanum majorana Leaf Extract); Dermican® LS 9837 (INCI: Glycerin (and) Water (and) Acetyl Tetrapeptide-9); Dermican® LS 9838 (INCI: Mannitol (and) Acetyl Tetrapeptide-9); Elestan® LS 9913 (INCI: Glycerin (and) Manilkara Multinervis Leaf Extract (and) Water); Elestan® LS 9879 (INCI: Manilkara Multinervis Leaf Extract (and) Maltodextrin); Eperuline® (INCI: Maltodextrin (and) Eperua falcata Bark Extract); Epigenist® (INCI: Maltodextrin (and) Voandzeia Subterranea Seed Extract); Hyalufix® (INCI: Water (and) Butylene Glycol (and) Alpinia Galanga Extract (and) Pentylene Glycol (and) Xanthan Gum (and) Caprylic/Capric Triglyceride), Hyaluronic Filling Spheres® (INCI: Ethylhexyl Palmitate (and) Trihydroxystearin (and) Sodium Hyaluronate); Linefactor® C (INCI: Water (and) Butylene Glycol (and) Pentylene Glycol (and) Hibiscus abelmoschus Extract (and) Xanthan Gum); Lox-Age® (INCI: Water (and) Cichorium intybus (Chicory) Leaf Extract (and) Hexylene Glycol (and) Caprylyl Glycol (and) Xanthan Gum); Lys'Iastine® V (INCI: Water (and) Butylene Glycol (and) Peucedanum graveolens (Dill) Extract (and) Pentylene Glycol (and) Xanthan Gum); Myoxinol® (INCI: Hydrolyzed Hibiscus Esculentus Extract (and) Dextrin); Neurobiox® (INCI: Water (and) Butylene Glycol (and) Pentylene Glycol (and) Achillea millefolium Extract (and) Xanthan Gum); Oligolin® (INCI: Hydrolyzed Linseed Extract); Perlaura® (INCI: Water (and) Polygonum Bistorta Root Extract (and) Hexylene Glycol (and) Caprylyl Glycol (and) Xanthan Gum); Phytokine® (INCI: Hydrolyzed Soy Protein (and) Butylene Glycol (and) Pentylene Glycol (and) 1,2-Hexanediol (and) Caprylyl Glycol); Proteasyl® LS 9818 (INCI: Water (and) Glycerin (and) Pisum Sativum (Pea) Extract); Proteasyl® LS 8951 (INCI: Pisum Sativum (Pea) Extract (and) Cyclodextrin); Shadownyl® (INCI: Water (and) Fucus Vesiculosus Extract (and) Hexylene Glycol (and) Caprylyl Glycol (and) Xanthan Gum); Slim-Excess® (INCI: Water (and) Butylene Glycol (and) Pentylene Glycol (and) Sodium Chloride (and) Hydrolyzed Rhodophycea Extract (and) Xanthan Gum); Speci'Men® (INCI: Glycerin (and) Water (and) Hydrolyzed Adansonia digitata Extract); Sqisandryl® (INCI: Schizandra Chinensis Fruit Extract); Syniorage® (INCI: Mannitol (and) Acetyl Tetrapeptide-11); Ultra Filling Sphreres® (INCI: Ethylhexyl Palmitate (and) Trihydroxystearin (and) Sodium Hyaluronate (and) Glucomannan); Vegeseryl® HGP (INCI: Water (and) Glycine soja (Soybean) Protein (and) Sodium Cocoyl Glutamate (and) Ethylhexylglycerin); Vit-A-Like® LS 9793 (INCI: Water (and) Vigna aconitifolia Seed Extract (and) Sodium Cocoyl Glutamate); Vit-A-Like® LS 9898 (INCI: Vigna aconitifolia Seed Extract (and) Maltodextrin); X-pressin® R (INCI: Water (and) Papain (and) Carbomer (and) Caprylyl Glycol (and) Algin).

Suitable active biogenic ingredients for improving the skin (skin perfection) are: AH-Care® (INCI: Water (and) Lactic Acid (and) Arginine); Beta-Hydroxyde® ACSD (INCI: Salicylic Acid (and) Acacia Senegal Gum); Betapur® (INCI: Water (and) Butylene Glycol (and) Peumus Boldus Leaf Extract (and) Pentylene Glycol (and) Xanthan Gum); Biophytex® (INCI: Water (and) Butylene Glycol (and) Panthenol (and) Escin (and) Glycerin (and) Ruscus Aculeatus Root Extract (and) Ammonium Glycyrrhizate (and) Centella Asiatica Leaf Extract (and) Hydrolyzed Yeast Protein (and) Calendula Officinalis Flower Extract); Lox-Age® (INCI: Water (and) Cichorium Intybus (Chicory) Leaf Extract (and) Hexylene Glycol (and) Caprylyl Glycol (and) Xanthan Gum); Mat-XS® Clinical (INCI: Water (and) Butylene Glycol (and) Pentylene Glycol (and) Sarcosine (and) Xanthan Gum); Neurobiox® (INCI: Water (and) Butylene Glycol (and) Pentylene Glycol (and) Achillea Millefolium Extract (and) Xanthan Gum; X-pressin® C (INCI: Water (and) Papain (and) Carbomer (and) Caprylyl Glycol (and) Algin).

Humectants

Humectants can bind water and hence prevent evaporation.

Suitable humectants are polyols having 2 to 10 carbon atoms and 2 to 6 hydroxyl groups, for example glycerol, sorbitol, propylene glycol, butylene glycol, polyethylene glycols or xylitol.

Insect Repellents

Examples of useful insect repellents include N,N-diethyl-m-toluamide, 1,2-pentanediol or ethyl 3-(N-n-butyl-N-acetylamino)propionate, which is sold under the Insect Repellent® 3535 name by Merck KGaA, and butyl acetylaminopropionates.

Tyrosine Inhibitors

Examples of useful tyrosine inhibitors which prevent the formation of melanin and find use in depigmenting agents include arbutin, ferulic acid, kojic acid, coumaric acid and ascorbic acid (vitamin C).

Perfume Oils

Perfume oils include mixtures of natural and synthetic odorants. Natural odorants are extracts from flowers (lily, lavender, rose, jasmine, neroli, ylang ylang), stems and leaves (geranium, patchouli, petitgrain), fruits (aniseed, coriander, caraway, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, for example civet and castoreum. Typical synthetic odorant compounds are products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include primarily the terpenes and balsams. However, preference is given to using mixtures of different odorants which together produce a pleasant scent note. Essential oils of relatively low volatility, which are mostly used as aroma components, are also suitable as perfume oils, e.g. sage oil, chamomile oil, clove oil, melissa oil, mint oil, cinnamon leaf oil, linden blossom oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillate, irotyl and floramate alone or in mixtures. Examples of suitable aromas include peppermint oil, spearmint oil, aniseed oil, star anise oil, caraway oil, eucalyptus oil, fennel oil, lemon oil, wintergreen oil, clove oil, menthol and the like.

Film Formers

Suitable film formers are, for example, chitosan, microcrystalline chitosan, quaternized chitosan, polyvinylpyrrolidone, vinylpyrrolidone-vinyl acetate copolymers, quaternary cellulose derivatives, collagen, hyaluronic acid and salts thereof and similar compounds.

Dyes

Dyes which can be used are the substances approved and suitable for cosmetic purposes, as listed, for example, in the publication “Kosmetische Fsrbemittel” [Cosmetic Colorants] from the Farbstoffkommission der Deutschen Forschungsgemeinschaft [Dyes Commission of the German Research Society], Verlag Chemie, Weinheim, 1984, pp. 81-106. Examples are cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). As a luminescent dye, it is also possible for luminol to be present. These dyes are usually used in concentrations of from 0.001% to 0.1% by weight, based on the total mixture.

Effect Pigments

Pigments may be present. The preferred particle size is 0.01 to 200 μm, especially 0.02 to 150 μm, more preferably 0.05 to 100 μm.

The pigments are colorants that are virtually insoluble in the application medium and may be inorganic or organic. Inorganic-organic mixed pigments are also possible. Preference is given to inorganic pigments.

The advantage of the inorganic pigments is their excellent photostability, weather stability and thermal stability. The inorganic pigments may be of natural origin, for example prepared from chalk, ochre, umber, green earth, burnt siena or graphite. The pigments may be white pigments, such as, for example, titanium dioxide or zinc oxide, black pigments, such as, for example, iron oxide black, colored pigments, such as, for example, ultramarine or iron oxide red, pearlescent pigments, metal effect pigments, pearlescent pigments and fluorescent or phosphorescent pigments, where preferably at least one pigment is a colored, non-white pigment.

Metal oxides, hydroxides and oxide hydrates, mixed-phase pigments, sulfur-containing silicates, metal sulfides, complex metal cyanides, metal sulfates, chromates and molybdates, and the metals themselves (bronze pigments) are suitable. Of particular suitability are titanium dioxide (Cl 77891), black iron oxide (Cl 77499), yellow iron oxide (Cl 77492), red and brown iron oxide (Cl 77491), manganese violet (Cl 77742), ultramarine (sodium aluminum sulfosilicates, Cl 77007, Pigment Blue 29), chromium oxide hydrate (Cl 77289), iron blue (ferric ferrocyanide, Cl 77510), carmine (cochineal).

Particular preference is given to pearlescent pigments and colored pigments based on mica or based on borosilicate, which are coated with a metal oxide or a metal oxychloride, such as titanium dioxide or bismuth oxychloride, and if appropriate further color-imparting substances, such as iron oxides, iron blue, ultramarine, carmine etc., and where the color can be determined by varying the layer thickness. Such pigments are sold, for example, under the Rona®, Colorona®, Dichrona® and Timiron® trade names by Merck or the Reflecks® Cloisonne®, Timica®, Chione®, Pearl-Glo® or Bi-Lite® trade names by BASF.

Organic pigments are, for example, the natural pigments sepia, gamboge, bone charcoal, Cassel brown, indigo, chlorophyll and other plant pigments.

Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

Especially suitable are, for instance, silica, silica, aluminates, aluminas, mica, salts, especially organic metal salts, metal oxides, e.g. titanium dioxide, optionally in combination with fluorescent or phosphorescent pigments.

Sensory Additives Examples of suitable sensory additives include Aluminium Starch, Octenylsuccinate, Tapoca, Dimethicone and Dimethicone Crosspolymer or Polymethyl silsequioxane.

Coolants

Examples of suitable coolants include ethanol, menthol and/or camphor.

The active cosmetic ingredients may be present in the base mixture in b1) in amounts of 0% to 50% by weight, preferably to 30% by weight, especially to 20% by weight—calculated as active substance. The cosmetic auxiliaries and additives—without water—be present in amounts of 0% to 25% by weight. Water may present in the base mixture in amounts of 0% to 75% by weight, preferably 0.5% to 70% by weight, according to the cosmetic application.

In one embodiment, b1) preferably comprises
0.5% to 75% by weight of at least one emollient,
1.0% to 30% by weight of at least one interface-active substance,
1.5% to 30% by weight of at least one rheology modifier,
0% to 50% by weight of at least one UV filter,
0% to 25% by weight of active cosmetic ingredients and/or cosmetic auxiliaries and additives
0% to 90% by weight of water,
under the condition that all constituents add up to 100% by weight.

Preferably, the base mixture i) comprising preferably at least one rheology modifier is produced by means of a solid oil mixture or a pre-emulsion, or ii) preferably not comprising any rheology modifier is produced by means of a liquid oil mixture. More preferably, the base mixture comprising at least one rheology modifier is produced by means of a pre-emulsion, preferably comprising at least one interface-active substance and at least one rheology modifier. In another preferred embodiment, the base mixture comprising at least one rheology modifier is produced by means of a solid oil mixture, preferably comprising at least one interface-active substance and at least one rheology modifier. In a further preferred embodiment, the base mixture not comprising any rheology modifier is produced by means of a liquid oil mixture, preferably comprising at least one interface-active substance.

Preferably, the base mixture in b1) comprises
10% to 60% by wt., preferably 25% to 58% by wt., of at least one emollient, 1% to 20% by wt., preferably 2% to 18% by wt., of at least one interface-active substance, preferably at least one nonionic and/or anionic interface-active substance,
0% to 20% by wt., preferably 0.1% to 16% by wt., of at least one rheology modifier,
5% to 50% by wt., preferably 10% to 40% by wt., of at least one UV filter,
0% to 25% by wt., preferably 0% to 20% by wt., of active cosmetic ingredients and/or cosmetic auxiliaries or additives and
0% to 50% by wt., preferably 0% to 35% by wt., of water,
based in each case on the total weight of the base mixture, preferably under the condition that all constituents add up to 100% by weight.

Advantageously, the base mixture in b1) comprises
10% to 60% by weight of emollient
3% to 30% by weight of interface-active substance, preferably anionic and nonionic 1.5% to 8% by weight of rheology modifier 5% to 50% by weight of UV filter
0% to 25% by weight of active cosmetic ingredients and/or cosmetic auxiliaries and additives and
0% to 40% by weight of water,
under the condition that all constituents add up to 100% by weight.

Advantageously, the base mixture in b1) comprises
20% to 50% by wt., preferably 25% to 45% by wt., of at least one emollient
3% to 20% by wt., preferably 5% to 15% by wt., of at least one interface-active substance, preferably nonionic
0.1% to 5% by wt., preferably 0.1% to 4% by wt., of at least one rheology modifier
10% to 30% by wt., preferably 12% to 25% by wt., of at least one UV filter
0% to 25% by wt., preferably 0% to 20% by wt., of active cosmetic ingredients and/or cosmetic auxiliaries and additives and
1% to 45% by wt., preferably 5% to 40% by wt., of water,
based in each case on the total weight of the base mixture, preferably under the condition that all constituents add up to 100% by weight.

In this context, the base mixture in b1) is preferably produced via a pre-emulsion. In the production of the pre-emulsion, preference is given to blending a portion A comprising the at least one UV filter with the at least one emollient. Separately, a portion B is made up, comprising the at least one interface-active substance, the at least one rheology modifier and water. Portions A and B are combined in order to obtain the base mixture in b1). Base mixtures that are produced by means of a pre-emulsion are especially suitable for production of sunscreens having moderate viscosity, for example milks.

Advantageously, the base mixture in b1) comprises
40% to 70% by wt., preferably 45% to 60% by wt., of at least one emollient
3% to 20% by wt., preferably 5% to 15% by wt., of at least one interface-active substance, preferably nonionic and/or anionic
3% to 25% by wt., preferably 5% to 20% by wt., of at least one rheology modifier
12% to 35% by wt., preferably 15% to 30% by wt., of at least one UV filter
0% to 25% by wt., preferably 0% to 20% by wt., of active cosmetic ingredients and/or cosmetic auxiliaries and additives and
0% to 40% by wt., preferably 0% to 35% by wt., of water,
based in each case on the total weight of the base mixture, preferably under the condition that all constituents add up to 100% by weight.

In this context, the base mixture in b1) is preferably produced via a solid oil mixture. In the production of the solid oil mixture, preference is given to blending a portion A comprising the at least one UV filter with the at least one emollient. Separately, a portion B is made up, comprising the at least one interface-active substance, the at least one rheology modifier and at least one emollient. Portions A and B are combined in order to obtain the base mixture in b1). Base mixtures that are produced by means of a solid oil mixture are especially suitable for production of sunscreens having relatively high viscosity, for example creams.

In the context of the present invention, a solid oil mixture has a melting point of 35° C. or more, preferably of greater than 45° C. More preferably, a solid oil mixture has a melting point of 35 to 100° C., especially of 45 to 90° C.

In the context of the present invention, the solid oil mixture is first heated before being emptied into the mixing device a) in order to assure sufficient flowability. In this context, the base mixture in b1) is heated preferably to more than 4° C., more preferably to more than 50° C. and especially to more than 60° C. Most preferably, the base mixture in b1) is heated to 40 to 120° C., more preferably to 50° C. to 100° C. and especially to 60° C. to 90° C.

Advantageously, the base mixture in b1) comprises
40% to 75% by wt., preferably 45% to 65% by wt., of at least one emollient
3% to 20% by wt., preferably 5% to 15% by wt., of at least one interface-active substance, preferably nonionic
no rheology modifier
15% to 50% by wt., preferably 30% to 40% by wt., of at least one UV filter
0% to 25% by wt., preferably 0% to 20% by wt., of active cosmetic ingredients and/or cosmetic auxiliaries and additives and
0% to 40% by wt., preferably 0% to 35% by wt., of water,
based in each case on the total weight of the base mixture, preferably under the condition that all constituents add up to 100% by weight.

In this context, the base mixture in b1) is preferably produced via a liquid oil mixture. In the production of the liquid oil mixture, preference is given to blending a portion A comprising the at least one UV filter with the at least one emollient. Separately, a portion B is made up, comprising the at least one interface-active substance. Portions A and B are combined in order to obtain the base mixture in b1). Base mixtures that are produced by means of a liquid oil mixture are especially suitable for production of sunscreens having low viscosity, for example fluids.

In the context of the present invention, a liquid oil mixture has a melting point of less than 35° C., preferably of less than 20° C. More preferably, a liquid oil mixture has a melting point of −10 to less than 35° C., especially of 5 to 30° C.

Particularly preferably, the base mixture for the production of a sunscreen in b1) comprises
10% to 60% by weight of at least one emollient selected from the group formed by esters of linear and/or branched C₆-C₁₈ fatty acids with linear C₆-C₂₂ fatty alcohols, esters of dicarboxylic acids with linear or branched alcohols having 1 to 22 carbon atoms and/or linear and branched C₆-C₂₂ fatty alcohol carbonates and especially the adipic acid, sebacic esters or malic esters of linear or branched alcohols having 1 to 18 carbon atoms, esters of benzoic acid with linear and/or branched C₆-C₂₂ alcohols, esters of hydroxycarboxylic acids with linear C₆-C₂₂ fatty alcohols
1% to 20% by weight of anionic interface-active surfactants selected from the group formed by alkyl polyethylene glycol ether citrates, salts of acyl glutamate, salts of mono- and dialkyl sulfosuccinates having 12 to 24 carbon atoms in the alkyl group, salts of alkyl sulfates and alkyl phosphates, and citric esters of glyceryl stearate
1% to 20% by weight of nonionic interface-active substances, preferably those from the group formed by polyglyceryl esters, sucrose esters, ethoxylated fatty alcohols and ethoxylated fatty acids and alkyl (oligo)glucosides
1.5% to 8% by weight of rheology modifiers, preferably selected from the group formed by hectorites and/or bentonites that have optionally been hydrophobically modified, polyacrylic acid and the alkali metal salts thereof, copolymers of polyacrylic acid and the alkali metal salts thereof, copolymers of (meth)acrylic acid and polyacrylate, where the polyether chain is terminated by a C8-C30-alkyl radical, copolymers of acrylamidomethylpropanesulfonic acid and alkali metal salts thereof, polysaccharides, fatty alcohols, glyceryl mono-, di- and/or tri-fatty acid esters and wax esters
5% to 50% by weight of UV filter
   0% to 25% by weight of active cosmetic ingredients and/or cosmetic auxiliaries and additives and
   0% to 20% by weight of water,
under the condition that the anionic and nonionic interface-active substance add up to 3% to not more than 30% by weight and all constituents add up to 100% by weight.

Most preferably, the base mixture for the production of a sunscreen in b1) comprises
10% to 60% by weight of at least one emollient selected from the group formed by esters of linear and/or branched C₆-C₁₈ fatty acids with linear C₆-C₂₂ fatty alcohols, esters of dicarboxylic acids with linear or branched alcohols having 1 to 22 carbon atoms and/or linear and branched C₆-C₂₂ fatty alcohol carbonates and especially the adipic acid esters, sebacic esters or malic esters of linear or branched alcohols having 1 to 18 carbon atoms, liquid mono-/di-/triglyceride mixtures based on C₆-C₁₈ fatty acids and esters of linear C₆-C₂₂ fatty acids with linear or branched C₆-C₂₂ fatty alcohols
1% to 20% by weight of nonionic interface-active substances, preferably those from the group formed by polyglyceryl esters, sucrose esters, ethoxylated fatty alcohols and ethoxylated fatty acids and alkyl (oligo)glucosides
0% to 16% by weight of rheology modifiers, preferably selected from the group formed by hectorites and/or bentonites that have optionally been hydrophobically modified, polyacrylic acid and the alkali metal salts thereof, copolymers of polyacrylic acid and the alkali metal salts thereof, copolymers of (meth)acrylic acid and polyacrylate, where the polyether chain is terminated by a C8-C30-alkyl radical, copolymers of acrylamidomethylpropanesulfonic acid and alkali metal salts thereof, polysaccharides, fatty alcohols, glyceryl mono-, di- and/or tri-fatty acid esters and wax esters
5% to 50% by weight of at least one UV filter
0% to 25% by weight of active cosmetic ingredients and/or cosmetic auxiliaries and additives and
0% to 35% by weight of water,
   under the condition that all constituents add up to 100% by weight.

Most preferably, the base mixture for the production of a sunscreen in b1) comprises
10% to 60% by weight of at least one emollient selected from the group formed by esters of linear and/or branched C₆-C₁₈ fatty acids with linear C₆-C₂₂ fatty alcohols, esters of dicarboxylic acids with linear or branched alcohols having 1 to 22 carbon atoms and/or linear and branched C₆-C₂₂ fatty alcohol carbonates and especially the adipic acid, sebacic esters or malic esters of linear or branched alcohols having 1 to 18 carbon atoms, liquid mono-/di-/triglyceride mixtures based on C₆-C₁₈ fatty acids and esters of linear C₆-C₂₂ fatty acids with linear or branched C₆-C₂₂ fatty alcohols
1% to 20% by weight of anionic interface-active surfactants selected from the group formed by alkyl polyethylene glycol ether citrates, salts of acyl glutamate, salts of mono- and dialkyl sulfosuccinates having 12 to 24 carbon atoms in the alkyl group, salts of alkyl sulfates and alkyl phosphates, and citric esters of glyceryl stearate
1% to 20% by weight of nonionic interface-active substances, preferably those from the group formed by polyglyceryl esters, sucrose esters, ethoxylated fatty alcohols and ethoxylated fatty acids and alkyl (oligo)glucosides
0% to 10% by weight of rheology modifiers, preferably selected from the group formed by hectorites and/or bentonites that have optionally been hydrophobically modified, polyacrylic acid and the alkali metal salts thereof, copolymers of polyacrylic acid and the alkali metal salts thereof, copolymers of (meth)acrylic acid and polyacrylate, where the polyether chain is terminated by a C8-C30-alkyl radical, copolymers of acrylamidomethylpropanesulfonic acid and alkali metal salts thereof, polysaccharides, fatty alcohols, glyceryl mono-, di- and/or tri-fatty acid esters and wax esters
5% to 50% by weight of at least one UV filter
0% to 25% by weight of active cosmetic ingredients and/or cosmetic auxiliaries and additives and
0% to 15% by weight of water,
   under the condition that the anionic and nonionic interface-active substances add up to 3% to not more than 30% by weight and all constituents add up to 100% by weight.

The cosmetic formulation produced by the method of the invention comprises the base mixture in b1) to an extent of at least 5% by weight and not more than 80% by weight, preferably at least 10% by weight and not more than 70% by weight, and especially at least 15% by weight and not more than 60% by weight—based on the cosmetic formulation.

More preferably, the sunscreen produced by the method of the invention comprises the base mixture in b1) to an extent of at least 10% by weight and not more than 50% by weight, preferably at least 15% by weight and not more than 40% by weight, and especially at least 20% by weight and not more than 36% by weight—based on the cosmetic formulation. Alternatively, the sunscreen produced by the method of the invention comprises the base mixture more preferably to an extent of at least 40% by weight and not more than 80% by weight, preferably at least 50% by weight and not more than 75% by weight, and especially at least 55% by weight and not more than 72% by weight—based on the cosmetic formulation.

Preferably, the base mixture in b1) has an SPF of 6 to 20, more preferably of 6 to 15. The SPF value can be obtained here by the simulation calculation of BASF sunscreen simulator (https://sunscreensimulator.basf.com/) or by the in vitro method described in the examples.

b2) Dispensing Vessel with Cosmetic Active Ingredients for Personalization

In the method of the invention, the cosmetic formulations are partly determined (“personalized”) by the end user in terms of their composition in accordance with the demand of the end user on the effect of the cosmetic formulation.

In one embodiment, the personalization can be obtained via the active cosmetic ingredients, or the active cosmetic ingredients are utilized for the personalization. If, for example, the end user has a demand or desire for a particularly moisturizing cosmetic formulation, this can be met by the method of the invention, in that, for example, a humectant is present in dispensing vessel b2). The demand or desire for a moisturizing sun milk formulation can be met by the method of the invention in that, for example, UV filters and humectants are present in dispensing vessel b2).

Accordingly, vessel b2) may comprise a multitude of active cosmetic ingredients and UV filters for personalization, according to the demand or desire of the end user. Preferably, the amount of active cosmetic ingredients and/or UV filters in formulations in b2) is at least 0.5% to not more than 30% by weight, preferably at least 1% to not more than 25% by weight and especially at least 1% to not more than 24% by weight—based on the cosmetic formulation. Preferably, the amount of active cosmetic ingredients and/or UV filters in b2) is at least 1% to not more than 15% by weight, preferably at least 2% to not more than 12% by weight and especially at least 3% to not more than 10% by weight—based on the cosmetic formulation. More preferably, the amount of UV filters in b2) is at least 0.5% to not more than 15% by weight, preferably at least 1% to not more than 10% by weight and especially at least 1% to not more than 8% by weight—based on the cosmetic formulation.

Suitable UV filters for b2) for personalization have already been described in the context of the UV filters for b1). Especially suitable for the formulations in b2) are water-dispersed UV filters. Most preferably, the dispensing vessel b2) comprises a UV filter-comprising formulation. The UV filter-comprising formulation preferably corresponds to the base mixture in dispensing vessel b1). Alternatively, the UV filter-comprising formulation preferably comprises at least one water-dispersed UV filter, preferably selected from the group consisting of Methylene Bis-Benzotriazolyl Tetramethylbutylphenol, Tris-Biphenyl Triazine, Bis-Ethylhexyloxyphenol Methoxyphenyl Triazine, Phenylene bis-diphenyltriazine, Bis-(Diethylaminohydroxybenzoyl Benzoyl) Piperazine and mixtures thereof.

Suitable active cosmetic ingredients for b2) for personalization have already been described in connection with the active cosmetic ingredients for b1), such as the active biogenic ingredients, secondary light stabilizers, self-tanning agents, insect repellents, antioxidants, film formers, sensory additives, effect pigments, swelling agents, tyrosine inhibitors (depigmenting agents), coolants, perfume oils, dyes, humectants and the like. Suitable compounds have been described by way of example in connection with the base mixture in b1), and explicit reference is hereby made thereto. Reference is hereby also made explicitly to the particularly suitable compounds and mixtures thereof that are described in b1).

If desired, it is also possible for b2) to comprise the auxiliaries and additives customarily used in cosmetics that are described in connection with the base mixture, such as water, stabilizers, solvents, solubilizers, preservatives, neutralizing agents and buffers, perfume oils, dyes, humectants, complexing agents and the like. Suitable compounds have been described by way of example in connection with the base mixture in b1), and explicit reference is hereby made thereto. Reference is hereby also made explicitly to the particularly suitable compounds and mixtures thereof that are described in b1).

A particular embodiment of the present invention is directed to cosmetic formulations for which the (end) user desires high proportions of emollients and/or active cosmetic ingredients. If the end user desires, for example, a face cream having a very high light protection factor higher than that achievable by the UV filters from the base mixture in b1), this can be achieved by further UV filters in b2). Suitable compounds for this purpose are those described under UV filters in connection with the base mixture described further up, and explicit reference is hereby made thereto. Particularly suitable are the further UV filters selected from the group formed by Ethylhexyl Methoxycinnamate, Bis-Ethylhexyloxyphenol Methoxyphenyl Triazine, Ethylhexyl Triazone, Methylene Bis-Benzotriazolyl Tetramethylbutylphenole and Diethylamino Hydroxy Benzoyl Hexyl Benzoate. If the (end) user desires particularly rich cosmetic formulations such as a face cream having a high proportion of emollients that is higher than achievable by the emollients from the base mixture in b1), this type of personalization can be achieved by further emollients in b2). Suitable emollients have been described by way of example in connection with the base mixture, and explicit reference is hereby made thereto. Particularly suitable emollients here too are those selected from the group formed by esters of C₂-C₁₂ dicarboxylic acids with linear or branched alcohols having 1 to 22 carbon atoms, carbonates of linear or branched C₆-C₂₂ fatty alcohols, such as Dicaprylyl Carbonate or dipropylheptyl carbonate, hydrocarbons such as undecane/tridecane, esters of linear C₆-C₂₂ fatty acids with linear or branched C₆-C₂₂ fatty alcohols, and Guerbet alcohols based on fatty alcohols having 6 to 18 carbon atoms.

In other words, the personalization in the context of the invention can be effected by the use of UV filters and/or active cosmetic ingredients, it being possible to have the active cosmetic ingredients in b1) and b2) or else in b2) only.

In a further embodiment of the present invention, the dispensing device b) has further dispensing vessels b3) and optionally b4), preferably the further dispensing vessel b3). According to the demand of the end user on the cosmetic formulation, the method may be executed in such a way that UV filters and/or active cosmetic ingredients or cosmetic auxiliaries and additives or else rheology modifiers are present in the dispensing vessels b3) and optionally b4). Suitable UV filters and active cosmetic ingredients for b3) and optionally b4) for personalization have already been described in connection with the active cosmetic ingredients for b1) and/or b2), such as the active biogenic ingredients, secondary light stabilizers, self-tanning agents, insect repellents, antioxidants, film formers, sensory additives, effect pigments, swelling agents, tyrosine inhibitors (depigmenting agents), coolants, perfume oils, dyes, humectants and the like. Suitable compounds have been described by way of example in connection with the base mixture, and explicit reference is hereby made thereto. Reference is hereby also made explicitly to the particularly suitable compounds and mixtures thereof that are described in b1).

In preferred embodiments, the formulation in b3) comprises a water-based formulation comprising at least one interface-active substance, preferably at least one anionic interface-active substance. The water-based formulation may further comprise at least one rheology modifier, UV filter and/or active cosmetic ingredients. In particular, the water-based formulation comprises at least one interface-active substance, preferably at least one anionic interface-active substance, and at least one rheology modifier. A corresponding water-based formulation comprises from 55% to 95% by weight, preferably from 60% to 90% by weight, more preferably from 70% to 90% by weight, of water, based on the total weight of the water-based formulation. Suitable interface-active substances, rheology modifiers, UV filters and active cosmetic ingredients have already been described for the base mixture in b1). A corresponding water-based formulation is preferably used in b3) when b1) comprises a base mixture that is produced by means of a liquid oil mixture. In this context, the dispensing device b), in b1), preferably has a base mixture comprising no rheology modifier, preferably produced by means of a liquid oil mixture, and, in b3), a water-based formulation comprising water, at least one interface-active substance, preferably an anionic interface-active substance, optionally a rheology modifier, optionally glycerol and optionally a UV filter.

Accordingly, by the method of the invention, it is possible to obtain personalization of the cosmetic formulation in that, for example, UV filters and/or active cosmetic ingredients are present in the dispensing vessels b3) and optionally b4). But it is also possible that these are present both in b1) and b2) and b3) and optionally b4) or else solely in b2) and b3) and optionally b4) or else solely in b1) and b2).

If desired, it is also possible for b3) and optionally b4) to comprise the auxiliaries and additives customarily used in cosmetics that are described in connection with b1) and/or b2), such as water, stabilizers, solvents, solubilizers, preservatives, neutralizing agents and buffers, perfume oils, dyes, humectants, complexing agents and the like. Suitable compounds have been described by way of example in connection with the base mixture, and explicit reference is hereby made thereto. Reference is hereby also made explicitly to the particularly suitable compounds and mixtures thereof that are described as base mixture.

In a further configuration, rheology modifiers may be present in vessels b3) and optionally b4). Suitable rheology modifiers are, for example, the rheology modifiers already mentioned in connection with the base mixture in b1) described further up, and explicit reference is hereby made thereto. Particularly suitable rheology modifiers here too are one or more compounds from the group formed by polyacrylic acids and the alkali metal salts thereof, copolymers of polyacrylic acid and the alkali metal salts thereof, copolymers of (meth)acrylic acid and polyether acrylates, where the polyether chain is terminated by a C8-C30-alkyl radical, copolymers of acrylamidomethylpropanesulfonic acid and the alkali metal salts thereof, the polysaccharides, the organic substances such as fatty alcohols, glyceryl mono-, di- and/or tri-fatty acid esters and wax esters, and the optionally chemically modified hectorites and bentonites. The presence of rheology modifiers in b3) and/or b4) can have an advantageous effect on the production method; in particular, the viscosities of the cosmetic formulation are controllable thereby. Suitable emollients are, for example, the emollients already mentioned further up in connection with the base mixture b1) or b2), to which explicit reference is hereby made, preferably those selected from the group formed by esters of C2-C12 dicarboxylic acids with linear or branched alcohols having 1 to 22 carbon atoms, carbonates of linear or branched C6-C22 fatty alcohols, such as Dicaprylyl Carbonate or dipropylheptyl carbonate, hydrocarbons such as undecane/tridecane, esters of linear C6-C22 fatty acids with linear or branched C6-C22 fatty alcohols, and Guerbet alcohols based on fatty alcohols having 6 to 18 carbon atoms. b3) and optionally b4) preferably additionally comprise emollients when large amounts thereof are desired, for example, for the production of skin lotions.

If required, the dispensing vessel b3) or b4) comprises active cosmetic ingredients for personalization, preferably in amounts of 0.01% to not more than 20% by weight, preferably at least 0.5% to not more than 15% by weight and especially at least 1% to not more than 10% by weight—based on the cosmetic formulation. This applies to each of the dispensing vessels, independently of one another.

In a very particular embodiment of the present invention, the dispensing device for the production of a sun milk formulation comprises three or four dispensing vessels, where b1) comprises a base mixture consisting of
10% to 60% by weight of at least one emollient, preferably selected from the group formed by esters of linear and/or branched C₆-C₁₈ fatty acids with linear C₆-C₂₂ fatty alcohols, esters of dicarboxylic acids with linear or branched alcohols having 1 to 22 carbon atoms and/or linear and branched C₆-C₂₂ fatty alcohol carbonates and especially the adipic acid, sebacic esters or malic esters of linear or branched alcohols having 1 to 18 carbon atoms, esters of benzoic acid with linear and/or branched C₆-C₂₂ alcohols, esters of hydroxycarboxylic acids with linear C₆-C₂₂ fatty alcohols
1% to 20% by weight of anionic interface-active surfactants, preferably selected from the group formed by alkyl polyethylene glycol ether citrates, salts of acyl glutamate, salts of mono- and dialkyl sulfosuccinates having 12 to 24 carbon atoms in the alkyl group, salts of alkyl sulfates and alkyl phosphates, and citric esters of glyceryl stearate
1% to 20% by weight of nonionic interface-active substance, preferably those from the group formed by polyglyceryl esters, sucrose esters, ethoxylated fatty alcohols and ethoxylated fatty acids and alkyl (oligo)glucosides
1.5% to 8% by weight of rheology modifiers, preferably selected from the group formed by hectorites and/or bentonites that have optionally been hydrophobically modified, polyacrylic acid and the alkali metal salts thereof, copolymers of polyacrylic acid and the alkali metal salts thereof, copolymers of (meth)acrylic acid and polyacrylate, where the polyether chain is terminated by a C8-C30-alkyl radical, copolymers of acrylamidomethylpropanesulfonic acid and alkali metal salts thereof, polysaccharides, fatty alcohols, glyceryl mono-, di- and/or tri-fatty acid esters and wax esters
5% to 50% by weight of UV filter,
   0% to 25% by weight of active cosmetic ingredients and/or cosmetic auxiliaries or additives and
   0% to 20% by weight of water
under the condition that the anionic and nonionic interface-active substance add up to 3% to not more than 30% by weight and all constituents add up to 100% by weight, and
b2) comprises UV filters and/or active cosmetic ingredients for personalization, cosmetic auxiliaries and additives, and emollients, preferably selected from the group formed by active biogenic ingredients, humectants, perfume oils, preservatives, UV filters, pH regulators/buffers, dyes, emollients and film formers, and b3) and optionally b4) comprise UV filters and/or further active cosmetic ingredients for personalization, auxiliaries and additives, and emollients, preferably selected from the group formed by active biogenic ingredients, humectants, perfume oils, preservatives, UV filters, pH regulators/buffers, dyes, emollients and film formers.

In a further particular embodiment of the present invention, the dispensing device for the production of a sunscreen formulation comprises three or four dispensing vessels, where b1) comprises a base mixture consisting of
10% to 60% by weight of at least one emollient, preferably selected from the group formed by triglycerides based on C₆-C₁₀ fatty acids, liquid mono-/di-/triglyceride mixtures based on C₆-C₁₈ fatty acids, esters of linear and/or branched C₆-C₁₈ fatty acids with linear C₁-C₂₂ fatty alcohols, esters of dicarboxylic acids with linear or branched alcohols having 1 to 22 carbon atoms and/or linear and branched C₁-C₂₂ fatty alcohol carbonates and especially the adipic acid esters, sebacic esters or malic esters of linear or branched alcohols having 1 to 18 carbon atoms, esters of benzoic acid with linear and/or branched C₁-C₂₂ alcohols, esters of hydroxycarboxylic acids with linear C₁-C₂₂ fatty alcohols
1% to 20% by weight of at least one nonionic interface-active substance, preferably those from the group formed by polyglyceryl esters, sucrose esters, ethoxylated fatty alcohols and ethoxylated fatty acids and alkyl (oligo)glucosides
0% to 16% by weight of at least one rheology modifier, preferably selected from the group formed by hectorites and/or bentonites that have optionally been hydrophobically modified, polyacrylic acids and the alkali metal salts thereof, copolymers of polyacrylic acid and the alkali metal salts thereof, copolymers of (meth)acrylic acid and polyether acrylates, where the polyether chain is terminated by a C8-C30-alkyl radical, copolymers of acrylamidomethylpropanesulfonic acid and alkali metal salts thereof, polysaccharides, fatty alcohols, glyceryl mono-, di- and/or tri-fatty acid esters and wax esters
5% to 50% by weight of at least one UV filter,
0% to 25% by weight of active cosmetic ingredients, cosmetic auxiliaries or additives and
0% to 20% by weight of water
under the condition that all constituents add up to 100% by weight, and
b2) comprises UV filters, optionally active cosmetic ingredients for personalization, cosmetic auxiliaries and additives, and emollients, preferably selected from the group formed by active biogenic ingredients, humectants, perfume oils, preservatives, secondary light stabilizers, pH regulators/buffers, dyes, emollients and film formers, and b3) and optionally b4) comprise further UV filters and/or active cosmetic ingredients for personalization, auxiliaries and additives, and emollients, preferably selected from the group formed by active biogenic ingredients, humectants, perfume oils, preservatives, UV filters, secondary light stabilizers, pH regulators/buffers, dyes, emollients and film formers.

The cosmetic formulations produced in accordance with the invention are preferably creams, gels, lotions, milk, sera, fluids, alcoholic and aqueous/alcoholic solutions, emulsions, wax/oil masses, pastes or ointments, which especially comprise water.

In the context of the present invention, water may be present in one or more of vessels b1), b2), b3) and/or optionally b4).

Preferably, the method of the invention is performed in a device that additionally has a water tank c) as well as a mixing device a) and a dispensing device b).

In the context of the present invention, various devices or apparatuses are suitable for performance of the method of the invention. Such devices are known from the prior art. For example, the capsule machine according to DE102016106040 A or EP2038189 B is a suitable device for performance of the method of the invention.

In one embodiment of the invention, it is preferable when the device additionally has a water tank c).

In the method of the invention, the contents of the dispensing vessels b1) and b2) and any b3) and optionally b4) are emptied into the mixing device a) and mixed with the water from the water tank c). Preferably, in the method of the invention, the contents from the dispensing vessels b1) and b2) and any b3) and optionally b4) are emptied into the mixing device a) and mixed with water at least in amounts of 30% by weight, preferably 35% to 80% by weight, more preferably 50% to 80% by weight—based on the cosmetic formulations—from the water tank c).

The amounts of water below 100% by weight—based on cosmetic formulations—may be present in one or more of the dispensing vessels that are emptied into the mixing device a).

Preferably, the mixing device a) comprises a vessel a1) with an optionally lowerable stirrer at the base. Advantageously, after the mixing, the stirrer at the base of the mixing vessel a1), i.e. after the production of the cosmetic formulation, is retracted and the mixing vessel is closed. Thus closed, it can be utilized as transportable storage vessel for the cosmetic formulations by the end user.

If desired, the dispensing vessels b1), b2), and optionally b3) and optionally b4) may be heated in a heating system d) before being emptied into the mixing vessel a1), preferably to temperatures up to 80° C. In general, it is advisable to conduct the method of the invention in such a way that the dispensing vessel b1) with the base mixture is heated. The process of the invention can alternatively be conducted solely at room temperature. According to the invention, first of all, the contents of the dispensing vessel b1) are emptied into the mixing device and mixed, preferably by means of a stirrer present in the base of the mixing vessel a1). Subsequently, the ingredients in dispensing vessels b2) and any b3) and optionally b4), preferably successively, are emptied into the mixing vessel a1) and stirred. If required, water may additionally be emptied into the mixing vessel a1) from the water tank c). If required, the water can be heated in a separate boiler. The contents of the dispensing vessels are especially expanded by pressure and emptied into the container with stirrer (mixing device a1). Preference is given to stirring in the container a1) at room temperature or while heating (max. 85° C.) and at up to 5000 revolutions/min for about 2-30 minutes. In particular, amounts of 20, 25 or 30 g of cosmetic formulation are produced by the method.

Preferably, in the context of the invention, the dispensing vessels b1), b2), any b3) and any b4) are capsules. For b1), preference is given to capsules having the following dimensions: Height: 3.5 to 4.5 cm and a diameter in the range from 3.8 to 4.2 cm (large capsule). The capsules for b2) may either have the dimensions of the large capsule or the following dimensions: Height 3.5 to 4.5 cm and diameter 2.2 to 2.6 cm (medium capsule). The capsules for b3) may have the dimensions of the large capsule or of the middle capsule or the following dimensions: Height 3.5 to 4.5 cm and a diameter in the range from 1.2 to 1.8 cm (small capsule), and the capsules for b4) may have the dimensions of the large or middle or small capsule, preferably the middle or small capsule, where the diameters of all capsules may be different on the two sides. More preferably, the capsules are made of aluminum or plastic. Suitable comparable capsules are known, for example, in association with coffee formulation. Particular preference is given to capsules that already have a device in the capsule interior that, in the form of a piston or plunger, is capable of emptying the contents of the capsules; for example, the capsules may be constructed like a piston syringe.

The device of WO2018/073541 is particularly suitable for the method of the invention, and reference is explicitly made thereto. WO2018/073541 works with a mixing system comprising
a tank for liquid (see water tank c), especially water;
a liquid pump connected between an outlet of the liquid tank and the inlet of a liquid feed;
a movable plate (see dispensing device b)) that can be controlled by rotation about a vertical axis and comprises at least two chambers designed to encompass one capsule each (see dispensing vessels b1) and b2)), and mechanisms capable of expelling the contents of each capsule by pressure from their corresponding chamber in which they are accommodated and emptying them into the mixing vessel a1)
a mixing device that firstly comprises a container (see a1) disposed beneath the movable plate, which is controlled in order to position the chamber comprising the capsule with the base mixture (see dispensing vessel b1) and the chamber comprising the capsule with the active cosmetic ingredient for personalization (see dispensing vessel b2) above the container for accommodating the contents of b1) and b2) that are expelled from their respective chambers, wherein the vessel a1) is in fluid connection with the outlet of the liquid feed, in order, especially after the expulsion of the contents of b1), and b2) and any b3) and/or b4) into the vessel, to introduce liquid into the vessel, and secondly a mixing element which is accommodated in the vessel a1) and is capable of producing the cosmetic formulation in this container from the mixture of the liquid (especially water from the water tank) and the base mixture in b1), the active cosmetic ingredients for personalization in b2) and any in b3) and/or in b4).

In the context of the present invention, the device of WO2018/073541 used for the method of the invention is as disclosed in the preferred embodiments and in the figures FIG. 1-FIG. 8; in particular,
the mixing element is a stirrer which is mounted at the base of the container a1) and which can be driven by a motor, especially an electric motor, mounted in the mixing system
the container a1) is a removable vessel closed by a base wall in order to enable the storage of the cosmetic formulation for later use,
the dispensing vessel b1), b2) and optionally b3) and/or b4) is a capsule comprising a cylindrical wall, a base wall with a central opening closed by a breakable lid, and a piston wall that forms a piston accommodated in the cylindrical wall
penetration of the lid of the capsule is achieved by means of a piston or a metal pin which is moved by means of a motor, especially an electric motor, and pressurized outflow of the contents of the capsule into the container a1)
movable plate for the capsules that can be brought through the vessel a1) in order to enable the outflow of the contents of the capsules into the container
the movable plate is controlled with rotation by means of a motor, especially an electric motor, such that the pressure expulsion devices can bring about the outflow of the contents of each capsule
it comprises a heating unit.

The mode of action of the device is especially in figures FIG. 1, FIG. 2 (dispensing device), FIG. 3 (outlet of the dispensing device for contents of the capsules into mixing system), FIG. 6 mixer for mixing vessel a1).

Such a device is available on the market, for example under the “Emuage®” name.

In the context of the present invention, such devices can produce cosmetic formulations in domestically customary amounts; if the end user possesses their own device, for example according to WO2018/073541, they are able to determine the site of production themselves; if a different private or else commercial person such as a drugstore should have such a device, the end user is able to partly determine the site of production. The same applies to the juncture of production.

The composition of the cosmetic formulations can be determined (“personalized”) by the end user according to the demands of the end user on the effect of the cosmetic formulations. Advantageously, the method of the invention is controlled by information or data that are transmitted electronically either to the end user or to the device, preferably by means of a QR code. Preferably, specific compositions of cosmetic formulations are assigned to the demands of the end users on the cosmetic formulations, for which a combination of capsules is recorded. This information or data is preferably recorded on an app.

Preferably in accordance with the invention, the method is run in such a way that the end user provides the dispensing device b) with the dispensing vessels b1), b2) and any b3) and optionally b4), preferably in accordance with the information or data transmitted to the end user by means of an app.

For the control of the method, it has been found to be particularly useful to provide information/data on the outer wall of the dispensing vessels, preferably in the form of a QR code. It is thus possible to control the method of the invention, preferably the process sequence (sequence of capsules), method parameters such as stirring speeds, method temperatures of the heating system for the dispensing vessels and or the juncture and amounts of water via the water tank by means of the information/data on the outer wall of the dispensing vessels.

The present invention further provides a dispensing vessel comprising a base mixture of at least one emollient, at least one surface-active substance, at least one UV filter and at least one rheology modifier or no rheology modifier for the production of domestically customary amounts of sunscreens that can be produced immediately in situ at the wish of the end user and are variable in terms of their composition, preferably usable for the method of the invention.

Preferably, the dispensing vessel comprises a base mixture comprising at least one emollient, at least one interface-active substance, at least one triazine derivative UV filter, at least one UV-A filter and at least one rheology modifier or no rheology modifier.

The dispensing vessel comprising a base mixture of at least one emollient, at least one interface-active substance, at least one UV filter and at least one rheology modifier or no rheology modifier may be used for the production of domestically customary amounts of cosmetic formulations (i.e. sunscreens) that can be produced immediately in situ at the wish of the end user and are variable in terms of their composition, preferably for the method of the invention, and the use of a dispensing vessel comprising UV filters and/or active cosmetic ingredients for personalization, preferably selected from the group formed by active biogenic ingredients, secondary light stabilizers, self-tanning agents, insect repellents, antioxidants, film formers, sensory additives, effect pigments, tyrosine inhibitors (depigmenting agents), coolants, perfume oils, dyes and humectants, for production of domestically customary amounts of cosmetic formulations that can be produced immediately in situ at the wish of the end user and are variable in terms of their composition, preferably for the method of the invention.

The base mixture in a dispensing vessel comprising at least one emollient, at least one surface-active substance, at least one UV filter and at least one rheology modifier or no rheology modifier may be used for the production of domestically customary amounts of cosmetic formulations that can be produced immediately in situ at the wish of the end user and are variable in terms of their composition, preferably for the method according to claim 1, and the use of UV filters and/or active cosmetic ingredients for personalization, preferably selected from the group formed by active biogenic ingredients, secondary light stabilizers, self-tanning agents, insect repellents, antioxidants, film formers, sensory additives, effect pigments, tyrosine inhibitors (depigmenting agents), coolants, perfume oils, dyes and humectants, for production of domestically customary amounts of cosmetic formulations that can be produced immediately in situ at the wish of the end user and are variable in terms of their composition, preferably for a method according to claim 1. The present invention finally provides for the use of a base mixture in a dispensing vessel comprising at least one emollient, at least one interface-active substance, at least one UV filter and at least one rheology modifier or no rheology modifier in combination with at least one UV filter, preferably at least one water-dispersed UV filter, and/or active cosmetic ingredients for personalization, where the active cosmetic ingredients are preferably selected from the group formed by active biogenic ingredients, secondary light stabilizers, self-tanning agents, insect repellents, antioxidants, film formers, sensory additives, effect pigments, tyrosine inhibitors (depigmenting agents), coolants, perfume oils, dyes and humectants, present in a further dispensing vessel, for the production of domestically customary amounts of cosmetic formulations (i.e. sunscreens) which immediately at the wish of the end user, preferably for the method of the invention according to claim 1.

### EXAMPLES

Capsules in the various sizes “big” (capacity 11.4 ml), “medium” (capacity 2.25 ml) and “small” (capacity 0.9 ml) were filled. The “big” capsule, corresponding to b1), comprised the base mixture of the corresponding sunscreen. Unless mentioned explicitly, the “medium” capsules comprised the cosmetic ingredients b2) needed for fulfillment of the personalized customer wishes (for example the “medium” capsules b2) a, b2) b or b2) c in example 1). The “medium” and/or “small” capsules comprised cosmetic ingredients that served for fulfillment of optional personalized customer wishes (capsules b3) and b4)).

A capsule machine according to international patent application WO2018073541 was utilized. For production, according to the personalized customer wishes, the capsules were inserted into the devices intended. After the machine had been switched on, the capsule contents were pushed into the container; according to the cosmetic formulation chosen, water was pumped in from the separate water tank. The type of capsule(s) to be selected and the amount of water were recorded in a program for operation of the capsule machine. The amount of water required was dispensed from a water tank. If required, the water can be heated in a separate boiler. The contents of the capsules inserted were expressed automatically and emptied into the container with stirrer (mixing device a). Stirring was conducted in the container a1) at room temperature and at up to 5000 revolutions/min for about 2-30 minutes. The exact method conditions were controlled automatically via the programming of the capsule machine. Different amounts of formulation were produced->20, 25 or 30 g.

### Examples 1.1 to 1.12: Sunscreen Milk SPF 30

In example 1, capsule b1) with the base mixture was used throughout with one of capsules b2) a, b and c. The choice of capsule b2) depends on the wishes of the end user. Each of these combinations b1) and b2) resulted in a sunscreen milk. It was also possible to further personalize each of these 3 combinations with one of the optional capsules b3). For example, capsules b1)+b2) a gave a preserved perfumed sun milk with a nourishing skinfeel and an SPF of 30.

Capsules b1)+b2)a+b3)a gave a preserved perfumed sun milk with a nourishing skinfeel and SPF30 and additional anti-aging effect.

Various personalized sunscreen milk formulations were produced.

TABLE 1
Compositions for examples 1.1 to 1.12

| | | Concentration | | |
|---|---|---|---|---|
| | | overall | | Amount |
| | | formulation in | | required |
| Capsule | Ingredient (chem. name) | % by wt. | Function | for 25 g |
| b1) | Di-n-Butyl adipate | 9 | Emollient | 2.25 |
| | Di-n-Octyl carbonate | 3.78 | Emollient | 0.945 |
| | Sodium Polyacrylate | 1.1 | Rheology | 0.275 |
| | | | modifier | |
| | Water, demin. | 0.3 | | 0.075 |
| | p-Methoxycinnamic acid 2- | 6 | UV filter | 1.5 |
| | ethylhexyl ester | | | |
| | 2,4-Diethylamino | 6 | UV filter | 1.5 |
| | Hydroxybenzoyl Hexyl Benzoate | | | |
| | 2,4,6-Trianilino-(p-carbo-2′- | 1.8 | UV filter | 0.45 |
| | Ethylhexyl-1′-oxy)-1,3,5-Triazine | | | |
| | 2,4-bis-{[4-(2-ethylhexyloxy)-2- | 1.5 | UV filter | 0.375 |
| | hydroxy]phenyl}-6-(4- | | | |
| | methoxyphenyl)-(1,3,5)-triazine | | | |
| | Polyethylene glycol (7) Dodecyl | 2.7 | Interface-active | 0.675 |
| | Ether Citrate | | substance | |
| | Polyglycerin poly-12- | 2.4 | Interface-active | 0.6 |
| | hydroxystearic acid ester | | substance | |
| | Dodecyl Glucoside | 0.3 | Interface-active | 0.075 |
| | | | substance | |
| | Sodium Stearoyl Glutamate | 0.2 | Interface-active | 0.05 |
| | | | substance | |
| | Triglyceryl Monocaprate | 0.1 | Interface-active | 0.025 |
| | | | substance | |
| | Triethanolamine | 0.6 | Neutralizing | 0.15 |
| | | | agent | |
| | Benzyldimethylstearylammonium | 0.42 | Rheology | 0.105 |
| | Hectorite | | modifier | |
| | 4-Methyl-1,3-dioxolan-2-one | 0.105 | Auxiliary | 0.026 |
| | Sodium Hydroxide (50% | 0.3 | Neutralizing | 0.075 |
| | solution) | | agent | |
| b2) a | Dipropylheptyl carbonate | 5 | Emollient | 1.25 |
| | 2-Phenoxyethanol, | 1 | Preservative | 0.25 |
| | Ethylhexylglycerin | | | |
| | Perfume 1 | 1 | Perfume oil | 0.25 |
| b2) b | Undecane, Tridecane | 5 | Emollient | 1.25 |
| | 2-Phenoxyethanol, | 1 | Preservative | 0.25 |
| | Ethylhexylglycerin | | | |
| | Perfume 2 | 1 | Perfume oil | 0.25 |
| b2) c | 2-Propylheptyl Octanoate | 5 | Emollient | 1.25 |
| | 2-Phenoxyethanol, | 1 | Preservative | 0.25 |
| | Ethylhexylglycerin | | | |
| | Perfume 3 | 1 | Perfume oil | 0.25 |
| optionally | | | | |
| b3) a | Water, Pentylene Glycol, | 1 | Active biogenic | 0.25 |
| | Hamamelis Virginiana leaf | | ingredient | |
| | extract, Xanthan gum, Caprylyl | | | |
| | Glycol | | | |
| b3) b | Glycerin | 2 | Humectant | 0.5 |
| b3) c | D-Panthenol, Water | 2 | Active biogenic | 0.5 |
| | | | ingredient | |
| Water | Water, demin. | ad 100 | | Make |
| tank | | | | up to |
| | | | | 25 g |

All sunscreen formulations were produced: b1)+b2) a, b1)+b2) b, b)+b2)c and b)+b2) a+b3) a, b1)+b2) b+b3)a, b1)+b2) c+b3) a and b1)+b2) a+b3) b, b1)+b2) b+b3)b, b1)+b2) c+b3) b and b1)+b2) a+b3) c, b1)+b2) b+b3)c, b1)+b2) c+b3)c.

All sun milk formulations produced were homogeneous and stable.

### Examples 2.1 to 2.12: Sunscreen Milk SPF 30

In example 2, capsule b1) was used throughout with one of capsules b2) a, b and c. The choice of capsule b2) depends on the wishes of the end user. Each of these combinations b) and b2) resulted in a sunscreen milk. It was also possible to further personalize each of these 3 combinations with one of the optional capsules b3). For example, capsules b1)+b2) a resulted a preserved perfumed sun milk with a nourishing skinfeel and an SPF of 30.

Capsules b1)+b2)a+b3)a gave a preserved perfume sun milk with a nourishing skinfeel and SPF30 and additional anti-aging effect.

Analogously to example 1, various personalized sunscreen milk formulations were produced.

TABLE 2
Compositions for examples 2.1 to 2.12

| | | Concentration | | |
|---|---|---|---|---|
| | | overall | | Amount |
| | | formulation in | | required |
| Capsule | Ingredient (chem. name) | % by wt. | Function | for 25 g |
| b1) | Di-n-Butyl adipate | 9 | Emollient | 2.25 |
| | Di-n-Octyl carbonate | 8.5 | Emollient | 2.125 |
| | Caprylic/capric triglyceride | 1.0 | Emollient | 0.25 |
| | Sodium Acrylates Copolymer | 1.0 | Rheology | 0.25 |
| | | | modifier | |
| | Water, demin. | 2.3 | | 0.575 |
| | p-Methoxycinnamic acid 2- | 6 | UV filter | 1.5 |
| | ethylhexyl ester | | | |
| | 2,4-Diethylamino | 6 | UV filter | 1.5 |
| | Hydroxybenzoyl Hexyl Benzoate | | | |
| | 2,4,6-Trianilino-(p-carbo-2′- | 1.8 | UV filter | 0.45 |
| | Ethylhexyl-1′-oxy)-1,3,5-Triazine | | | |
| | 2,4-bis-{[4-(2-ethylhexyloxy)-2- | 1.5 | UV filter | 0.375 |
| | hydroxy]phenyl}-6-(4- | | | |
| | methoxyphenyl)-(1,3,5)-triazine | | | |
| | Polyethylene glycol (7) Dodecyl | 2.7 | Interface-active | 0.675 |
| | Ether Citrate | | substance | |
| | Polyglycerine poly-12- | 2.4 | Interface-active | 0.6 |
| | hydroxystearic acid ester | | substance | |
| | Dodecyl Glucoside | 0.3 | Interface-active | 0.075 |
| | | | substance | |
| | Sodium Stearoyl Glutamate | 0.2 | Interface-active | 0.05 |
| | | | substance | |
| | Triethanolamine | 0.6 | Neutralizing | 0.15 |
| | | | agent | |
| | Benzyldimethylstearylammonium | 1.2 | Rheology | 0.3 |
| | Hectorite | | modifier | |
| | 4-Methyl-1,3-dioxolan-2-one | 0.3 | Auxiliary | 0.075 |
| b1) | Sodium Hydroxide (50% | 0.2 | Neutralizing | 0.05 |
| | solution) | | agent | |
| b2) a | Dipropylheptyl carbonate | 5 | Emollient | 1.25 |
| | 2-Phenoxyethanol, | 1 | Preservative | 0.25 |
| | Ethylhexylglycerin | | | |
| | Perfume 1 | 1 | Perfume oil | 0.25 |
| b2) b | Undecane, Tridecane | 5 | Emollient | 1.25 |
| | 2-Phenoxyethanol, | 1 | Preservative | 0.25 |
| | Ethylhexylglycerin | | | |
| | Perfume 2 | 1 | Perfume oil | 0.25 |
| b2) b | | | | |
| b2) c | 2-Propylheptyl Octanoate | 5 | Emollient | 1.25 |
| | 2-Phenoxyethanol, | 1 | Preservative | 0.25 |
| | Ethylhexylglycerin | | | |
| | Perfume 3 | 1 | Perfume oil | 0.25 |
| optionally | | | | |
| b3) a | Water, Pentylene Glycol, | 1 | Active biogenic | 0.25 |
| | Hamamelis Virginiana leaf | | ingredient | |
| | extract, Xanthan gum, Caprylyl | | | |
| | Glycol | | | |
| b3) b | Glycerin | 2 | Humectant | 0.5 |
| b3) c | D-Panthenol, Water | 2 | Active biogenic | 0.5 |
| | | | ingredient | |
| Water | Water, demin. | ad 100 | | Make |
| tank | | | | up to |
| | | | | 25 g |

All sun milk formulations produced were homogeneous and stable.

### Example 3: Sunscreen Milk SPF 15

Analogously to example 1, various sunscreen milks were produced, with personalization of capsule b1) with one of capsules b2) and optionally with one of the further capsules b3) and b4).

TABLE 3
Compositions for example 3

| | | Concentration | | |
|---|---|---|---|---|
| | | overall | | Amount |
| | | formulation in | | required |
| Capsule | Ingredient (chem. name) | % by wt. | Function | for 25 g |
| b1) | Di-n-Butyl adipate | 4.5 | Emollient | 1.35 |
| | Undecane, Tridecane | 6.0 | Emollient | 1.8 |
| | C8-C18 Alkyl Ester | 2.0 | Emollient | 0.6 |
| | Di-n-Octyl carbonate | 2.5 | Emollient | 0.75 |
| | Dodecyl glucoside | 0.9 | Interface- | 0.27 |
| | | | active | |
| | | | substance | |
| | Polyglycerine poly-12- | 1.95 | Interface- | 0.585 |
| | hydroxystearic acid ester | | active | |
| | | | substance | |
| | Triglyceryl Monocaprate | 0.1 | Interface- | 0.03 |
| | | | active | |
| | | | substance | |
| | Polyethylene glycol (7) Dodecyl | 1.35 | Interface- | 0.405 |
| | Ether Citrate | | active | |
| | | | substance | |
| | Sodium Polyacrylate | 1.1 | Rheology | 0.33 |
| | | | modifier | |
| | p-Methoxycinnamic acid 2- | 2.85 | UV filter | 0.855 |
| | ethylhexyl ester | | | |
| | 2,4-Diethylamino | 2.85 | UV filter | 0.855 |
| | Hydroxybenzoyl Hexyl Benzoate | | | |
| b1) | 2,4,6-Trianilino-(p-carbo-2′- | 0.9 | UV filter | 0.27 |
| | Ethylhexyl-1′-oxy)-1,3,5-Triazine | | | |
| | 2,4-bis{[4-(2-ethylhexyloxy)-2- | 0.75 | UV filter | 0.225 |
| | hydroxy]phenyl}-6-(4- | | | |
| | methoxyphenyl)-(1,3,5)-triazine | | | |
| | Benzyldimethylstearylammonium | 0.24 | Rheology | 0.072 |
| | Hectorite | | modifier | |
| | Glycerin | 0.75 | Humectant | 0.225 |
| | 2-Phenoxyethanol, | 1.0 | Preservative | 0.3 |
| | Phenylmethanol, | | | |
| | Ethylhexylglycerin | | | |
| | Phenylpropanol, 1,2- | 1.0 | Preservation | 0.3 |
| | Propanediol, 1,2-Octanediol, | | aid | |
| | Tocopherol | | | |
| | 4-Methyl-1,3-dioxolan-2-one | 0.06 | Auxiliary | 0.018 |
| | Triethanolamine | 0.3 | Neutralizing | 0.09 |
| | | | agent | |
| | Water, demin. | 0.15 | | 0.045 |
| b2) a | Water, pentylene Glycol, | 1.0 | Active biogenic | 0.3 |
| | Hamamelis Virginiana leaf | | ingredient | |
| | extract, Xanthan gum, Caprylyl | | | |
| | Glycol | | | |
| | Glycerin | 2.0 | Humectant | 0.6 |
| | Phenylpropanol, 1,2- | 0.5 | Preservation | 0.15 |
| | Propanediol, 1,2-Octanediol, | | aid | |
| | Tocopherol | | | |
| b2) b | Glycerin | 3.00 | Humectant | 0.9 |
| b2) c | D-Panthenol | 2.00 | Active biogenic | 0.6 |
| | | | ingredient | |
| | Glycerin | 2.00 | Humectant | 0.6 |
| optionally: | | | | |
| b3) a | Water, pentylene Glycol, | 1.0 | Active biogenic | 0.3 |
| | Hamamelis Virginiana leaf | | ingredient | |
| | extract, Xanthan gum, Caprylyl | | | |
| | Glycol | | | |
| | Glycerin | 2.0 | Humectant | 0.6 |
| | Phenylpropanol, 1,2- | 0.5 | Preservation | 0.15 |
| | Propanediol, 1,2-Octanediol, | | aid | |
| | Tocopherol | | | |
| b3) b | Glycerin | 3.00 | Humectant | 0.9 |
| b3) c | D-Panthenol | 2.00 | Active biogenic | 0.6 |
| | | | ingredient | |
| | Glycerin | 2.00 | Humectant | 0.6 |
| optionally | | | | |
| b4) a | Perfume 1 | 0.40 | Perfume oil | 0.12 |
| | C8-C18 Alkyl Ester | 2.00 | Emollient | 0.6 |
| b4) b | Perfume 2 | 0.20 | Perfume oil | 0.06 |
| | C8-C18 Alkyl Ester | 2.00 | Emollient | 0.6 |
| b4) c | Perfume 3 | 0.20 | Perfume oil | 0.06 |
| | C8-C18 Alkyl Ester | 2.00 | Emollient | 0.6 |
| Water | Water, demin. | ad 100 | | Make |
| tank | | | | up to |
| | | | | 30 g |

All sunscreen milk formulations produced were homogeneous and stable.

### Example 4: Sunscreen Milk SPF 15

Analogously to example 1, various sunscreen milk formulations were produced, with personalization of capsule b1) with one of capsules b2) and optionally with one of the further capsules b3) and b4).

TABLE 4
Compositions for example 4

| | | Concentration | | |
|---|---|---|---|---|
| | | overall | | Amount |
| | | formulation in | | required |
| Capsule | Ingredient (chem. name) | % by wt. | Function | for 25 g |
| b1) | Di-n-Butyl adipate | 4.5 | Emollient | 1.35 |
| | Undecane, Tridecane | 6.0 | Emollient | 1.8 |
| | C8-C18 Alkyl Ester | 2.0 | Emollient | 0.6 |
| | Di-n-Octyl carbonate | 4.25 | Emollient | 1.275 |
| | Caprylic/capric triglyceride | 1.0 | Emollient | 0.3 |
| | Dodecyl glucoside | 0.15 | Interface- | 0.045 |
| | | | active | |
| | | | substance | |
| | Polyglycerine poly-12- | 1.2 | Interface- | 0.36 |
| | hydroxystearic acid ester | | active | |
| | | | substance | |
| | Polyethylene glycol (7) Dodecyl | 1.35 | Interface- | 0.405 |
| | Ether Citrate | | active | |
| | | | substance | |
| | Sodium Stearoyl Glutamate | 0.2 | Interface- | 0.06 |
| | | | active | |
| | | | substance | |
| b1) | Sodium Acrylates Copolymer | 1.0 | Rheology | 0.3 |
| | | | modifier | |
| | p-Methoxycinnamic acid 2- | 2.85 | UV filter | 0.855 |
| | ethylhexyl ester | | | |
| | 2,4-Diethylamino | 2.85 | UV filter | 0.855 |
| | Hydroxybenzoyl Hexyl Benzoate | | | |
| | 2,4,6-Trianilino-(p-carbo-2′- | 0.9 | UV filter | 0.27 |
| | Ethylhexyl-1′-oxy)-1,3,5-Triazine | | | |
| | 2,4-bis{[4-(2-ethylhexyloxy)-2- | 0.75 | UV filter | 0.225 |
| | hydroxy]phenyl}-6-(4- | | | |
| | methoxyphenyl)-(1,3,5)-triazine | | | |
| | Benzyldimethylstearylammonium | 0.6 | Rheology | 0.18 |
| | Hectorite | | modifier | |
| | 2-Phenoxyethanol, | 1.0 | Preservative | 0.3 |
| | Phenylmethanol, | | | |
| | Ethylhexylglycerin | | | |
| | Phenylpropanol, 1,2- | 1.0 | Preservation | 0.3 |
| | Propanediol, 1,2-Octanediol, | | aid | |
| | Tocopherol | | | |
| b1) | 4-Methyl-1,3-dioxolan-2-one | 0.15 | Auxiliary | 0.045 |
| | Triethanolamine | 0.3 | Neutralizing | 0.09 |
| | | | agent | |
| | Sodium Hydroxide (50%) | 0.2 | Neutralizing | 0.06 |
| | | | agent | |
| | Water, demin. | 2.15 | | 0.645 |
| b2) a | Water, pentylene Glycol, | 1.0 | Active biogenic | 0.3 |
| | Hamamelis Virginiana leaf | | ingredient | |
| | extract, Xanthan gum, Caprylyl | | | |
| | Glycol | | | |
| | Glycerin | 2.0 | Humectant | 0.6 |
| | Phenylpropanol, 1,2- | 0.5 | Preservative | 0.15 |
| | Propanediol, 1,2-Octanediol, | | aid | |
| | Tocopherol | | | |
| b2) b | Glycerin | 3.00 | Humectant | 0.9 |
| b2) c | D-Panthenol | 2.00 | Active biogenic | 0.6 |
| | | | ingredient | |
| | Glycerin | 2.00 | Humectant | 0.6 |
| optionally: | | | | |
| b3) a | Water, pentylene Glycol, | 1.0 | Active biogenic | 0.3 |
| | Hamamelis Virginiana leaf | | ingredient | |
| | extract, Xanthan gum, Caprylyl | | | |
| | Glycol | | | |
| | Glycerin | 2.0 | Humectant | 0.6 |
| | Phenylpropanol, 1,2- | 0.5 | Preservative | 0.15 |
| | Propanediol, 1,2-Octanediol, | | aid | |
| | Tocopherol | | | |
| b3) b | Glycerin | 3.00 | Humectant | 0.9 |
| b3) c | D-Panthenol | 2.00 | Active biogenic | 0.6 |
| | | | ingredient | |
| | Glycerin | 2.00 | Humectant | 0.6 |
| optionally | | | | |
| b4) a | Perfume 1 | 0.40 | Perfume oil | 0.12 |
| | C8-C18 Alkyl Ester | 2.00 | Emollient | 0.6 |
| b4) b | Perfume 2 | 0.20 | Perfume oil | 0.06 |
| b4) b | C8-C18 Alkyl Ester | 2.00 | Emollient | 0.6 |
| b4) c | Perfume 3 | 0.20 | Perfume oil | 0.06 |
| b4) c | C8-C18 Alkyl Ester | 2.00 | Emollient | 0.6 |
| Water | Water, demin. | ad 100 | | Make |
| tank | | | | up to |
| | | | | 30 g |

All sunscreen milk formulations produced were homogeneous and stable.

### Example 5: Sunscreen Milk SPF 40 or 50

Analogously to example 1, various sunscreen formulations were produced, with personalization of capsule b1) with capsule b2) and optionally with one of the further capsules b3) and b4).

Capsule b2) once again comprises a mixture comprising UV filters when a sun milk with an SPF of 50 rather than 40 is desired.

TABLE 5
Compositions for example 5

| | | Concentration | | |
|---|---|---|---|---|
| | | overall | | Amount |
| | | formulation in | | required |
| Capsule | Ingredient (chem. name) | % by wt. | Function | for 25 g |
| b1) | Di-n-Butyl adipate | 12.9 | Emollient | 2.58 |
| | Di-n-Octyl carbonate | 8.5 | Emollient | 1.7 |
| | Caprylic/capric triglyceride | 0.5 | Emollient | 0.1 |
| | Sodium Acrylates Copolymer | 0.5 | Rheology | 0.1 |
| | | | modifier | |
| | Polyethylene glycol (7) Dodecyl | 3.87 | Interface- | 0.774 |
| | Ether Citrate | | active | |
| | | | substance | |
| | Polyglycerine poly-12- | 3.44 | Interface- | 0.688 |
| | hydroxystearic acid ester | | active | |
| | | | substance | |
| | Dodecyl Glucoside | 0.43 | Interface- | 0.086 |
| | | | active | |
| | | | substance | |
| | Sodium Stearoyl Glutamate | 0.2 | Interface- | 0.04 |
| | | | active | |
| | | | substance | |
| | Water, demin. | 1.43 | | 0.286 |
| | Benzyldimethylstearylammonium | 1.2 | Rheology | 0.24 |
| | Hectorite | | modifier | |
| | 4-Methyl-1,3-dioxolan-2-one | 0.3 | Auxiliary | 0.06 |
| | p-Methoxycinnamic acid 2- | 8.2 | UV filter | 1.64 |
| | ethylhexyl ester | | | |
| | 2,4-Diethylamino | 8.2 | UV filter | 1.64 |
| | Hydroxybenzoyl Hexyl Benzoate | | | |
| | 2,4,6-Trianilino-(p-carbo-2′- | 2.58 | UV filter | 0.516 |
| | Ethylhexyl-1′-oxy)-1,3,5-Triazine | | | |
| | 2,4-bis{[4-(2-ethylhexyloxy)-2- | 2.0 | UV filter | 0.4 |
| | hydroxy]phenyl}-6-(4- | | | |
| | methoxyphenyl)-(1,3,5)-triazine | | | |
| | Sodium Hydroxide (50% | 0.4 | Neutralizing | 0.08 |
| | solution) | | agent | |
| | Triethanolamine | 0.86 | Neutralizing | 0.172 |
| | | | agent | |
| b2) | Di-n-Butyl adipate | 2.1 | Emollient | 0.42 |
| | Polyethylene glycol (7) Dodecyl | 0.63 | Interface- | 0.126 |
| | Ether Citrate | | active | |
| | | | substance | |
| | Polyglycerine poly-12- | 0.56 | Interface- | 0.112 |
| | hydroxystearic acid ester | | active | |
| | | | substance | |
| | Dodecyl Glucoside | 0.07 | Interface- | 0.014 |
| | | | active | |
| | | | substance | |
| | p-Methoxycinnamic acid 2- | 1.4 | UV filter | 0.28 |
| | ethylhexyl ester | | | |
| | 2,4-Diethylamino | 1.3 | UV filter | 0.26 |
| | Hydroxybenzoyl Hexyl Benzoate | | | |
| | 2,4,6-Trianilino-(p-carbo-2′- | 0.42 | UV filter | 0.084 |
| | Ethylhexyl-1′-oxy)-1,3,5-Triazine | | | |
| | 2,4-bis{[4-(2-ethylhexyloxy)-2- | 0.35 | UV filter | 0.07 |
| | hydroxylphenyl}-6-(4- | | | |
| | methoxyphenyl)-(1,3,5)-triazine | | | |
| | Triethanolamine | 0.14 | Neutralizing | 0.028 |
| | | | agent | |
| | Water, demin. | 0.07 | | 0.014 |
| optionally | Dipropylheptyl carbonate | 5 | Emollient | 1.25 |
| b3) a | 2-Phenoxyethanol, | 1 | Preservative | 0.25 |
| | Ethylhexylglycerin | | | |
| | Perfume 1 | 1 | Perfume oil | 0.25 |
| b3) a | Undecane, Tridecane | 5 | Emollient | 1.25 |
| b3) b | 2-Phenoxyethanol, | 1 | Preservative | 0.25 |
| | Ethylhexylglycerin | | | |
| | Perfume 2 | 1 | Perfume oil | 0.25 |
| | 2-Propylheptyl Octanoate | 5 | Emollient | 1.25 |
| b3) c | 2-Phenoxyethanol, | 1 | Preservative | 0.25 |
| | Ethylhexylglycerin | | | |
| | Perfume 3 | 1 | Perfume oil | 0.25 |
| optionally | Water, pentylene Glycol, | 1 | Active biogenic | 0.25 |
| | Hamamelis Virginiana leaf | | ingredient | |
| | extract, Xanthan gum, Caprylyl | | | |
| | Glycol | | | |
| b4) a | Glycerin | 2.0 | Humectant | 0.5 |
| b4) b | D-Panthenol | 2.0 | Active biogenic | 0.5 |
| | | | ingredient | |
| b4) c | Water, demin. | ad 100 | | Make |
| | | | | up to |
| | | | | 20 g |
| Water | | | | |
| tank | | | | |

All sun milk formulations produced were homogeneous and stable.

### Example 6: SPF 30 Sunscreen Formulation

In example 6, various sunscreen formulations were produced, with personalization of capsule b1) throughout with one of capsules b2) and with one of the further capsules b3) and optionally with one of capsules b4).

TABLE 6
Compositions for example 6

| | | Concentration | | |
|---|---|---|---|---|
| | | overall | | Amount |
| | | formulation in | | required |
| Capsule | Ingredient (chem. name) | % by wt. | Function | for 25 g |
| b1) | C12-15 alkyl benzoates | 7.0 | Emollient | 2.1 |
| | C8-C18 Alkyl Ester | 3.0 | Emollient | 0.9 |
| | Di-n-Octyl carbonate | 7.6 | Emollient | 2.28 |
| | Dodecyl glucoside | 0.75 | Interface-active | 0.225 |
| | | | substance | |
| | Polyglycerine poly-12- | 0.75 | Interface-active | 0.225 |
| | hydroxystearic acid ester | | substance | |
| | Triglyceryl Monocaprate | 0.1 | Interface-active | 0.03 |
| | | | substance | |
| | Sodium Polyacrylate | 1.0 | Rheology | 0.3 |
| | | | modifier | |
| | p-Methoxycinnamic Acid 2- | 6.0 | UV filter | 1.8 |
| | Ethylhexyl Ester | | | |
| | 2,4-bis{[4-(2-ethylhexyloxy)-2- | 3.0 | UV filter | 0.9 |
| | hydroxylphenyl}-6-(4- | | | |
| | methoxyphenyl)-(1,3,5)-triazine | | | |
| | Benzyldimethylstearylammonium | 0.2 | Rheology | 0.06 |
| | Hectorite | | modifier | |
| | Glycerin | 0.75 | Humectant | 0.225 |
| | 2-Phenoxyethanol, | 1.0 | Preservative | 0.3 |
| | Phenylmethanol, | | | |
| | Ethylhexylglycerin | | | |
| | Phenylpropanol, 1,2- | 1.0 | Preservative aid | 0.3 |
| | Propanediol, 1,2-Octanediol, | | | |
| | Tocopherol | | | |
| | 4-Methyl-1,3-dioxolan-2-one | 0.1 | Auxiliary | 0.03 |
| | Water, demin. | 0.75 | | 0.045 |
| b2) | 2,2′-Methylene-bis-(6-(2H- | 7.0 | UV filter | 2.1 |
| | Benzotriazole-2-yl)-4-(1,1,3,3- | | | |
| | Tetramethylbutyl)-phenol, | | | |
| | Water, | | | |
| | Decyl Glucoside, Propylene | | | |
| | Glycol, Xanthan Gum | | | |
| | Water, demin. | 0.5 | | 0.15 |
| b3) a | D-Panthenol | 2.0 | Active biogenic | 0.6 |
| | | | ingredient | |
| | Glycerin | 2.0 | Humectant | 0.6 |
| b3) b | 2-Methyl-1,4,5,6- | 1.0 | Active biogenic | 0.3 |
| | tetrahydropyrimidine-4- | | ingredient | |
| | carboxylic acid | | | |
| | Water, demin. | 5.0 | | 1.5 |
| | Phenylpropanol, 1,2- | 0.5 | Preservative aid | 0.15 |
| | Propanediol, 1,2-Octanediol, | | | |
| | Tocopherol | | | |
| b3) c | 3-α-D-Galacturonic Acid | 0.3 | Active biogenic | 0.09 |
| | Trisaccharide | | ingredient | |
| | Water, demin. | 5.0 | | 1.5 |
| | Propylene glycol | 2.0 | Humectant | 0.6 |
| optionally | | | | |
| b4) a | Perfume 1 | 0.4 | Perfume oil | 0.12 |
| | C8-C18 Alkyl Ester | 2.0 | Emollient | 0.6 |
| b4) b | Perfume 2 | 0.2 | Perfume oil | 0.06 |
| b4) b | C8-C18 Alkyl Ester | 2.0 | Emollient | 0.6 |
| b4) c | Perfume 3 | 0.2 | Perfume oil | 0.06 |
| | C8-C18 Alkyl Ester | 2.0 | Emollient | 0.6 |
| Water | Water, demin. | ad 100 | | Make |
| tank | | | | up to |
| | | | | 30 g |

All sunscreen formulations produced were homogeneous and stable.

### Example 7: SPF 15, 20 or 30 Sunscreen Milk

In example 7, various sunscreen formulations were produced, with personalization of capsule b) either with one of capsules b2) and with optionally one of the further capsules b3) and optionally with one of capsules b4) or with a capsule b3) and optionally a further capsule b4).

Capsule b2) either again comprises the mixture as in b1) or another UV filter mixture. Rather than a further UV filter mixture, the contents of capsule b1) may also be used directly for personalization with at least one other capsule (here b3) and/or b4)).

Capsules b1), b2)a and b2)b were provided in the “big” size. Capsule b2)c was provided in the “medium” size. If used, capsules b3) and b4) were provided in the “small” or “medium” size.

TABLE 7
Compositions for example 7;

| | | Concentration | | |
|---|---|---|---|---|
| | | in overall | | Amount |
| | Ingredient | formulation in | | required |
| Capsule | (chem. name) | % by wt. | Function | for 30 g |
| | Portion A | | | |
| b1) | Di-n-Butyl adipate | 5.0 | Emollient | 1.5 |
| | Di-n-Octyl carbonate | 5.0 | Emollient | 1.5 |
| | Glycerides, mixed coco, | 3.0 | Emollient | 0.9 |
| | decanoyl and octanoyl | | | |
| | Ethylhexyl triazone | 1.5 | UV filter | 0.45 |
| | Diethylhexyl butamido | 1.5 | UV filter | 0.45 |
| | triazone | | | |
| | Bis-ethylhexyloxyphenol | 1.0 | UV filter | 0.3 |
| | methoxyphenyl triazine | | | |
| | 2,4-Diethylamino | 3.0 | UV filter | 0.9 |
| | Hydroxybenzoyl Hexyl | | | |
| | Benzoate | | | |
| | 2-Phenoxyethanol, | 0.5 | Preservative | 0.15 |
| | Ethylhexylglycerin | | | |
| | Portion B (pre-emulsion) | | | |
| | Sucrose Polystearate | 1.5 | Interface-active | 0.45 |
| | (and) Cetyl Palmitate | | substance | |
| | Lauryl Glucoside (and) | 1.5 | Interface-active | 0.45 |
| | Polyglyceryl-2 | | substance | |
| | Dipolyhydroxystearate | | | |
| | Water | 10.5 | | 3.15 |
| | Xanthan Gum | 0.15 | Rheology modifier | 0.045 |

optional, if at least one of capsules b3) and b4) is used

| | Portion A | | | |
|---|---|---|---|---|
| b2) a | Di-n-Butyl adipate | 5.0 | Emollient | 1.5 |
| | Di-n-Octyl carbonate | 5.0 | Emollient | 1.5 |
| | Glycerides, mixed coco, | 3.0 | Emollient | 0.9 |
| | decanoyl and octanoyl | | | |
| | Ethylhexyl triazone | 1.5 | UV filter | 0.45 |
| | Diethylhexyl butamido | 1.5 | UV filter | 0.45 |
| | triazone | | | |
| | Bis-ethylhexyloxyphenol | 1.0 | UV filter | 0.3 |
| | methoxyphenyl triazine | | | |
| | 2,4-Diethylamino | 3.0 | UV filter | 0.9 |
| | Hydroxybenzoyl Hexyl | | | |
| | Benzoate | | | |
| | 2-Phenoxyethanol, | 0.5 | Preservative | 0.15 |
| | Ethylhexylglycerin | | | |
| | Portion B (pre-emulsion) | | | |
| | Sucrose Polystearate | 1.5 | Interface-active | 0.45 |
| | (and) Cetyl Palmitate | | substance | |
| | Lauryl Glucoside (and) | 1.5 | Interface-active | 0.45 |
| | Polyglyceryl-2 | | substance | |
| | Dipolyhydroxystearate | | | |
| | Water | 10.5 | | 3.15 |
| | Xanthan Gum | 0.15 | Rheology modifier | 0.045 |
| b2) b | Methylene Bis- | 6.0* | Water-dispersed | 1.8 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 6.0* | Water-dispersed | 1.8 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 5.0 | | 1.5 |
| b2) c | Bis-Ethylhexyloxyphenol | 5.0** | Water-dispersed | 1.5 |
| | | | UV filter | |
| | Methoxyphenyl | | | |
| | Triazine (and) | | | |
| | Acrylates/C12-22 Alkyl | | | |
| | Methacrylate | | | |
| | Copolymer | | | |
| b2) d | Methylene Bis- | 3.0* | Water-dispersed | 1.8 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 3.0* | Water-dispersed | 1.8 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 4.0 | | 1.2 |
| optionally | | | | |
| b3) a | Dipropylheptyl | 5 | Emollient | 1.5 |
| | carbonate | | | |
| | Perfume 1 | 1 | Perfume oil | 0.3 |
| b3) b | Coco-Caprylate | 1.7 | Emollient | 0.51 |
| | Perfume 1 | 0.3 | Perfume oil | 0.09 |
| b3) c | Propylheptyl caprylate | 1.7 | Emollient | 0.51 |
| | Perfume 1 | 0.3 | Perfume oil | 0.09 |
| optionally | | | | |
| b4) a | Water, pentylene | 1 | Active biogenic | 0.3 |
| | Glycol, Hamamelis | | ingredient | |
| | Virginiana leaf extract, | | | |
| | Xanthan gum, Caprylyl | | | |
| | Glycol | | | |
| b4) b | Glycerin | 2.0 | Humectant | 0.6 |
| b4) c | D-Panthenol | 2.0 | Active biogenic | 0.6 |
| | | | ingredient | |
| b4) d | 1,2-Hexanediol | 5.0 | Humectant | 1.5 |
| Water tank | Water, demin. | ad 100 | | Make up to 30 g |

*means that the amount is based on the mixture comprising the UV filter, with the UV filter accounting for 50% by weight of the mixture;
**means that the amount is based on the mixture comprising the UV filter, where the UV filter accounts for 20% by weight of the mixture

All sun milk formulations produced were homogeneous and stable at room temperature, 4° C. and 40′ C.

The SPF values of mixtures are reported hereinafter. The SPF values were ascertained using the BASF sunscreen simulator calculation. Mixtures that were produced without an additional UV-containing capsule b2) had an SPF of 15. Mixtures that were produced with capsule b2)a had an SPF of 30. Mixtures that were produced with capsule b2)b had an SPF of 30. Mixtures that were produced with capsule b2)c had an SPF of 20. Mixtures that were produced with capsule b2)d, according to the BASF sunscreen simulator calculation, had an SPF of 30. The SPF values of the mixtures comprising b1) and b1)+b2)a were likewise tested in vitro. A comparison of the in vitro values with the simulated values shows that the simulation is an excellent approximation to the SPF value (cf. table 13).

### Example 8: SPF 50 and 50+Sunscreen Milk

Analogously to example 7, various sunscreen formulations were produced, with personalization of capsule b1) with capsule b2) and optionally with one of the further capsules b3) and b4).

Capsule b2) either again comprises the mixture as in b1) or another UV filter mixture.

Capsules b1), b2)a, b2)b, b3)a and b3)b were provided in the “big” size. Capsule b3)c and b4)d was provided in the “medium” size. If used, the other capsules were provided in the “small” or “medium” size.

TABLE 8
Compositions for example 8; *means that the amount is based on the mixture
comprising the UV filter, with the UV filter accounting for 50% by weight of the mixture;
**means that the amount is based on the mixture comprising the UV filter, where the
UV filter accounts for 20% by weight of the mixture

| | | Concentration | | |
|---|---|---|---|---|
| | | in overall | | Amount |
| | | formulation in | | required |
| Capsule | Ingredient (chem. name) | % by wt. | Function | for 30 g |
| b1) | Portion A | | | |
| | Di-n-Butyl adipate | 5.0 | Emollient | 1.5 |
| | Di-n-Octyl carbonate | 5.0 | Emollient | 1.5 |
| | Glycerides, mixed coco, | 3.0 | Emollient | 0.9 |
| | decanoyl and octanoyl | | | |
| | Ethylhexyl triazone | 1.5 | UV filter | 0.45 |
| | Diethylhexyl butamido | 1.5 | UV filter | 0.45 |
| | triazone | | | |
| | Bis- | 1.0 | UV filter | 0.3 |
| | ethylhexyloxyphenol | | | |
| | methoxyphenyl triazine | | | |
| | 2,4-Diethylamino | 3.0 | UV filter | 0.9 |
| | Hydroxybenzoyl Hexyl | | | |
| | Benzoate | | | |
| | 2-Phenoxyethanol, | 0.5 | Preservative | 0.15 |
| | Ethylhexylglycerin | | | |
| | Portion B (pre-emulsion) | | | |
| | Sucrose Polystearate | 1.5 | Interface-active | 0.45 |
| | (and) Cetyl Palmitate | | substance | |
| | Lauryl Glucoside (and) | 1.5 | Interface-active | 0.45 |
| | Polyglyceryl-2 | | substance | |
| | Dipolyhydroxystearate | | | |
| | Water | 10.5 | | 3.15 |
| | Xanthan Gum | 0.15 | Rheology modifier | 0.045 |
| b2) a | Portion A | | | |
| | Di-n-Butyl adipate | 5.0 | Emollient | 1.5 |
| | Di-n-Octyl carbonate | 5.0 | Emollient | 1.5 |
| | Glycerides, mixed coco, | 3.0 | Emollient | 0.9 |
| | decanoyl and octanoyl | | | |
| | Ethylhexyl triazone | 1.5 | UV filter | 0.45 |
| | Diethylhexyl butamido | 1.5 | UV filter | 0.45 |
| | triazone | | | |
| | Bis- | 1.0 | UV filter | 0.3 |
| | ethylhexyloxyphenol | | | |
| | methoxyphenyl triazine | | | |
| | 2,4-Diethylamino | 3.0 | UV filter | 0.9 |
| | Hydroxybenzoyl Hexyl | | | |
| | Benzoate | | | |
| | 2-Phenoxyethanol, | 0.5 | Preservative | 0.15 |
| | Ethylhexylglycerin | | | |
| | Portion B (pre-emulsion) | | | |
| | Sucrose Polystearate | 1.5 | Interface-active | 0.45 |
| | (and) Cetyl Palmitate | | substance | |
| | Lauryl Glucoside (and) | 1.5 | Interface-active | 0.45 |
| | Polyglyceryl-2 | | substance | |
| | Dipolyhydroxystearate | | | |
| | Water | 10.5 | | 3.15 |
| | Xanthan Gum | 0.15 | Rheology modifier | 0.045 |
| b2) b | Methylene Bis- | 6.0* | Water-dispersed | 1.8 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 6.0* | Water-dispersed | 1.8 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 5.0 | | 1.5 |
| optionally | | | | |
| b3) a | Methylene Bis- | 3.0* | Water-dispersed | 0.9 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 3.0* | Water-dispersed | 0.9 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 4.0 | | 1.2 |
| b3) b | Methylene Bis- | 6.0* | Water-dispersed | 1.8 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 6.0* | Water-dispersed | 1.8 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 5.0 | | 1.5 |
| b3) c | Bis- | 5.0** | Water-dispersed | 1.5 |
| | Ethylhexyloxyphenol | | UV filter | |
| | Methoxyphenyl | | | |
| | Triazine (and) | | | |
| | Acrylates/C12-22 Alkyl | | | |
| | Methacrylate | | | |
| | Copolymer | | | |
| optionally | | | | |
| b4) a | Water, pentylene | 1 | Active biogenic | 0.3 |
| | Glycol, Hamamelis | | ingredient | |
| | Virginiana leaf extract, | | | |
| | Xanthan gum, Caprylyl | | | |
| | Glycol | | | |
| b4) b | Glycerin | 2.0 | Humectant | 0.6 |
| b4) c | D-Panthenol | 2.0 | Active biogenic | 0.6 |
| | | | ingredient | |
| b4) d | Bis- | 5.0** | Water-dispersed | 1.5 |
| | Ethylhexyloxyphenol | | UV filter | |
| | Methoxyphenyl | | | |
| | Triazine (and) | | | |
| | Acrylates/C12-22 Alkyl | | | |
| | Methacrylate | | | |
| | Copolymer | | | |
| Water tank | Water, demin. | ad 100 | | Make up |
| | | | | to 30 g |

All sun milk formulations produced were homogeneous and stable.

The SPF values of mixtures are reported hereinafter. The SPF values were ascertained using the BASF sunscreen simulator calculation. Mixtures that were produced additionally with capsule b2)a and b3)a had an SPF of 50. Mixtures that were produced additionally with capsule b2)a, b3)b and b4)d had an SPF of 50+(>60). Mixtures that were produced additionally with capsule b2)b and b3)c had an SPF of 50. The SPF values of the mixtures comprising b1)+b2)a+b3)a and b1)+b2)a+b3)b+b4)d were likewise tested in vitro. A comparison of the in vitro values with the simulated values shows that the simulation is an excellent approximation to the SPF value (cf. table 13).

### Example 9: SPF 15, 20, 30 and 50 Sunscreen Cream

Analogously to example 7, various sunscreen formulations were produced. By contrast with the production according to example 7, capsule b1) first had to be heated to 75° C.

Capsules b1)a, b1)b, b2)b and b2)c were provided in the “big” size. Capsules b2)a and b3)a was provided in the “medium” size. If used, the other capsules were provided in the “small” or “medium” size.

TABLE 9
Compositions for example 9; *means that the amount is based on the mixture
comprising the UV filter, with the UV filter accounting for 50% by weight of
the mixture;
**means that the amount is based on the mixture comprising the UV filter, where
the UV filter accounts for 20% by weight of the mixture.

| | | Concentration | | |
|---|---|---|---|---|
| | | in overall | | Amount |
| | | formulation in | | required |
| Capsule | Ingredient (chem. name) | % by wt. | Function | for 30 g |
| b1) a | Portion A | | | |
| | Di-n-Butyl adipate | 5.0 | Emollient | 1.5 |
| | Di-n-Octyl carbonate | 5.0 | Emollient | 1.5 |
| | Glycerides, mixed coco, | 3.0 | Emollient | 0.9 |
| | decanoyl and octanoyl | | | |
| | Ethylhexyl triazone | 1.5 | UV filter | 0.45 |
| | Diethylhexyl butamido | 1.5 | UV filter | 0.45 |
| | triazone | | | |
| | Bis- | 1.0 | UV filter | 0.3 |
| | ethylhexyloxyphenol | | | |
| | methoxyphenyl triazine | | | |
| | 2,4-Diethylamino | 3.0 | UV filter | 0.9 |
| | Hydroxybenzoyl Hexyl | | | |
| | Benzoate | | | |
| | 2-Phenoxyethanol, | 1.0 | Preservative | 0.3 |
| | Ethylhexylglycerin | | | |
| | Portion B (solid oil mixture) | | | |
| | Cetylstearyl alcohol | 2.5 | Rheology modifier | 0.75 |
| | Disodium Cetearyl | 0.7 | Interface-active | 0.21 |
| | Sulfosuccinate | | substance | |
| | Cetearyl Glucoside | 2.0 | Interface-active | 0.6 |
| | (and) Cetearyl Alcohol | | substance | |
| | Myristyl myristate | 2.5 | Emollient | 0.75 |
| b1) b | Portion A | | | |
| | Di-n-Butyl adipate | 5.0 | Emollient | 1.5 |
| | Di-n-Octyl carbonate | 5.0 | Emollient | 1.5 |
| | Glycerides, mixed coco, | 3.0 | Emollient | 0.9 |
| | decanoyl and octanoyl | | | |
| | Ethylhexyl triazone | 1.5 | UV filter | 0.45 |
| | Diethylhexyl butamido | 1.5 | UV filter | 0.45 |
| | triazone | | | |
| | Bis- | 1.0 | UV filter | 0.3 |
| | ethylhexyloxyphenol | | | |
| | methoxyphenyl triazine | | | |
| | 2,4-Diethylamino | 3.0 | UV filter | 0.9 |
| | Hydroxybenzoyl Hexyl | | | |
| | Benzoate | | | |
| | 2-Phenoxyethanol, | 0.5 | Preservative | 0.3 |
| | Ethylhexylglycerin | | | |
| | Portion B (solid oil mixture) | | | |
| | Cetylstearyl alcohol | 5.0 | Rheology modifier | 1.5 |
| | Disodium Cetearyl | 0.7 | Interface-active | 0.21 |
| | Sulfosuccinate | | substance | |
| | Cetearyl Glucoside | 2.0 | Interface-active | 0.6 |
| | (and) Cetearyl Alcohol | | substance | |
| | Myristyl myristate | 5.0 | Emollient | 1.5 |
| b2) a | Bis- | 5.0** | Water-dispersed | 1.5 |
| | Ethylhexyloxyphenol | | UV filter | |
| | Methoxyphenyl | | | |
| | Triazine (and) | | | |
| | Acrylates/C12-22 Alkyl | | | |
| | Methacrylate | | | |
| | Copolymer | | | |
| b2) b | Methylene Bis- | 3.0* | Water-dispersed | 0.9 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 3.0* | Water-dispersed | 0.9 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 4.0 | | 1.2 |
| b2) c | Methylene Bis- | 6.0* | Water-dispersed | 1.8 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 6.0* | Water-dispersed | 1.8 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 5.0 | | 1.5 |
| optionally | | | | |
| b3) a | Bis- | 5.0** | Water-dispersed | 1.5 |
| | Ethylhexyloxyphenol | | UV filter | |
| | Methoxyphenyl | | | |
| | Triazine (and) | | | |
| | Acrylates/C12-22 Alkyl | | | |
| | Methacrylate | | | |
| | Copolymer | | | |
| b3) b | Coco-Caprylate | 1.7 | Emollient | 0.51 |
| | Perfume 1 | 0.3 | Perfume oil | 0.09 |
| optionally | | | | |
| b4) a | Water, pentylene | 1 | Active biogenic | 0.3 |
| | Glycol, Hamamelis | | ingredient | |
| | Virginiana leaf extract, | | | |
| | Xanthan gum, Caprylyl | | | |
| | Glycol | | | |
| b4) b | Glycerin | 2.0 | Humectant | 0.6 |
| b4) c | D-Panthenol | 2.0 | Active biogenic | 0.6 |
| | | | ingredient | |
| Water tank | Water, demin. | ad 100 | | Make up |
| | | | | to 30 g |

All sunscreen cream formulations produced were homogeneous and stable.

The SPF values of mixtures produced are reported hereinafter. The SPF values were ascertained using the BASF sunscreen simulator calculation. Mixtures that were produced with capsule b1)a or b1)b had an SPF of 15. Mixtures that were produced additionally with capsule b2)a had an SPF of 20. Mixtures that were produced additionally with capsule b2)b had an SPF of 30. Mixtures that were produced additionally with capsules b2)c and b3)a had an SPF of 50. The SPF values were likewise tested in vitro. A comparison of the in vitro values with the simulated values shows that the simulation is an excellent approximation to the SPF value (cf. table 13).

### Example 10: SPF 10, 15, 25, 30 and 50 Sunscreen Cream as Foundation

Analogously to example 9, various sunscreen formulations suitable as foundation were produced.

Capsules b1), b2)b, b2)c and b3)a were provided in the “big” size. Capsule b2)a was provided in the “medium” size. If used, the other capsules were provided in the “small” or “medium” size.

TABLE 10
Compositions for example 10; *means that the amount is based on the mixture
comprising the UV filter, with the UV filter accounting for 50% by weight of the
mixture;
**means that the amount is based on the mixture comprising the UV filter, where
the UV filter accounts for 20% by weight of the mixture.

| | | Concentration | | |
|---|---|---|---|---|
| | | in overall | | |
| | | formulation in | | Amount required |
| Capsule | Ingredient (chem. name) | % by wt. | Function | for 30 g |
| b1) a | Portion A | | | |
| | Di-n-Butyl adipate | 4.0 | Emollient | 1.2 |
| | Di-n-Octyl carbonate | 4.0 | Emollient | 1.2 |
| | Ethylhexyl triazone | 1.0 | UV filter | 0.3 |
| | Diethylhexyl butamido | 1.5 | UV filter | 0.45 |
| | triazone | | | |
| | 2,4-Diethylamino | 2.5 | UV filter | 0.75 |
| | Hydroxybenzoyl Hexyl | | | |
| | Benzoate | | | |
| | 2-Phenoxyethanol, | 1.0 | Preservative | 0.3 |
| | Ethylhexylglycerin | | | |
| | Portion B (solid oil mixture) | | | |
| | Cetylstearyl alcohol | 2.5 | Rheology modifier | 0.75 |
| | Disodium Cetearyl | 0.7 | Interface-active | 0.21 |
| | Sulfosuccinate | | substance | |
| | Cetearyl Glucoside | 2.0 | Interface-active | 0.6 |
| | (and) Cetearyl Alcohol | | substance | |
| | Myristyl myristate | 2.5 | Emollient | 0.75 |
| | Titanium dioxide /Cl | 9.0 | Pigment | 2.7 |
| | 77891 | | | |
| | Yellow iron oxide / Cl | 2.25 | Pigment | 0.68 |
| | 77492 | | | |
| | Red iron oxide / Cl | 0.85 | Pigment | 0.26 |
| | 77491 | | | |
| | Black iron oxide /Cl | 0.45 | Pigment | 0.14 |
| | 77499 | | | |
| b2) a | Bis- | 5.0** | Water-dispersed | 1.5 |
| | Ethylhexyloxyphenol | | UV filter | |
| | Methoxyphenyl | | | |
| | Triazine (and) | | | |
| | Acrylates/C12-22 Alkyl | | | |
| | Methacrylate | | | |
| | Copolymer | | | |
| b2) b | Methylene Bis- | 3.0* | Water-dispersed | 0.9 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 3.0* | Water-dispersed | 0.9 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 4.0 | | 1.2 |
| b2) c | Methylene Bis- | 6.0* | Water-dispersed | 1.8 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 6.0* | Water-dispersed | |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 5.0 | | 1.5 |
| optionally | | | | |
| b3) a | Methylene Bis- | 3.0* | Water-dispersed | 0.9 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 3.0* | Water-dispersed | 0.9 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 4.0 | | 1.2 |
| b3) b | Coco-Caprylate | 1.7 | Emollient | 0.51 |
| | Perfume 1 | 0.3 | Perfume oil | 0.09 |
| optionally | | | | |
| b4) a | Water, pentylene | 1 | Active biogenic | 0.3 |
| | Glycol, Hamamelis | | ingredient | |
| | Virginiana leaf extract, | | | |
| | Xanthan gum, Caprylyl | | | |
| | Glycol | | | |
| b4) b | Glycerin | 2.0 | Humectant | 0.6 |
| b4) c | D-Panthenol | 2.0 | Active biogenic | 0.6 |
| | | | ingredient | |
| Water tank | Water, demin. | ad 100 | | Make up to 30 g |

All suncream formulations produced were homogeneous and stable.

The SPF values of mixtures are reported hereinafter. The SPF values were ascertained using the BASF sunscreen simulator calculation. Mixtures that were produced with capsule b.) had an SPF of 10. Mixtures that were produced additionally with capsule b2)a had an SPF of 15. Mixtures that were produced additionally with capsule b2)b had an SPF of 25. Mixtures that were produced additionally with capsule b2)c had an SPF of 30. Mixtures that were produced additionally with capsules b2)c and b3)a had an SPF of 50. The SPF value of the mixture comprising b1) was likewise tested in vitro. A comparison of the in vitro value with the simulated value shows that the simulation is an excellent approximation to the SPF value (cf. table 13), with the in vitro value actually being higher.

### Example 11: SPF 15, 30, 50 and 50+ Sunscreen Fluids

Analogously to example 7, various sunscreen formulations were produced. By contrast with the production according to example 7, capsule b3) was used throughout.

Capsules b1), b2)a, b4)a and b4)b were provided in the “big” size. Capsule b3) was provided in the “big” size. If used, the other capsules were provided in the “small” or “medium” size.

TABLE 11
Compositions for example 11; *means that the amount is based on the mixture
comprising the UV filter, with the UV filter accounting for 50% by weight of
the mixture

| | | Concentration | | |
|---|---|---|---|---|
| | | in overall | | |
| | | formulation in | | Amount required |
| Capsule | Ingredient (chem. name) | % by wt. | Function | for 30 g |
| b1) | Portion A | | | |
| | Di-n-Butyl adipate | 5.0 | Emollient | 1.5 |
| | Di-n-Octyl carbonate | 5.0 | Emollient | 1.5 |
| | Glycerides, mixed coco, | 3.0 | Emollient | 0.9 |
| | decanoyl and octanoyl | | | |
| | Ethylhexyl triazone | 1.5 | UV filter | 0.45 |
| | Diethylhexyl butamido | 1.5 | UV filter | 0.45 |
| | triazone | | | |
| | Bis- | 1.0 | UV filter | 0.3 |
| | ethylhexyloxyphenol | | | |
| | methoxyphenyl triazine | | | |
| | 2,4-Diethylamino | 3.0 | UV filter | 0.9 |
| | Hydroxybenzoyl Hexyl | | | |
| | Benzoate | | | |
| | 2-Phenoxyethanol, | 0.5 | Preservative | 0.15 |
| | Ethylhexylglycerin | | | |
| | Portion B (liquid oil mixture) | | | |
| | Polyglyceryl-2 | 2.0 | Interface-active | 0.6 |
| | Dipolyhydroxystearate | | substance | |
| optionally | | | | |
| b2) a | Portion A | | | |
| | Di-n-Butyl adipate | 5.0 | Emollient | 1.5 |
| | Di-n-Octyl carbonate | 5.0 | Emollient | 1.5 |
| | Glycerides, mixed coco, | 3.0 | Emollient | 0.9 |
| | decanoyl and octanoyl | | | |
| | Ethylhexyl triazone | 1.5 | UV filter | 0.45 |
| | Diethylhexyl butamido | 1.5 | UV filter | 0.45 |
| | triazone | | | |
| | Bis- | 1.0 | UV filter | 0.3 |
| | ethylhexyloxyphenol | | | |
| | methoxyphenyl triazine | | | |
| | 2,4-Diethylamino | 3.0 | UV filter | 0.9 |
| | Hydroxybenzoyl Hexyl | | | |
| | Benzoate | | | |
| | 2-Phenoxyethanol, | 0.5 | Preservative | 0.15 |
| | Ethylhexylglycerin | | | |
| | Portion B (liquid oil mixture) | | | |
| | Polyglyceryl-2 | 2.0 | Interface-active | 0.6 |
| | Dipolyhydroxystearate | | substance | |
| b2) b | Coco-Caprylate | 1.7 | Emollient | 0.51 |
| | Perfume 1 | 0.3 | Perfume oil | 0.09 |
| b3) | Sodium Lauryl Glucose | 1.5 | Interface-active | 0.45 |
| | Carboxylate (and) | | substance | |
| | Lauryl Glucoside | | | |
| | Sodium Polyacrylate | 1.0 | Rheology modifier | 0.3 |
| | (and) Dicaprylyl | | | |
| | Carbonate (and) | | | |
| | Polyglyceryl-3 Caprate | | | |
| | Xanthan Gum | 0.2 | Rheology modifier | 0.06 |
| | Water | 25.0 | | 7.5 |
| optionally | | | | |
| b4) a | Methylene Bis- | 3.0* | Water-dispersed | 0.9 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 3.0* | Water-dispersed | 0.9 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 4.0 | | 1.2 |
| b4) b | Methylene Bis- | 6.0* | Water-dispersed | 1.8 |
| | Benzotriazolyl | | UV filter | |
| | Tetramethylbutylphenol | | | |
| | (and) Aqua (and) Decyl | | | |
| | Glucoside (and) | | | |
| | Propylene Glycol (and) | | | |
| | Xanthan Gum | | | |
| | Tris-Biphenyl Triazine | 6.0* | Water-dispersed | 1.8 |
| | (and) Aqua (and) Decyl | | UV filter | |
| | Glucoside (and) | | | |
| | Butylene Glycol (and) | | | |
| | Disodium Phosphate | | | |
| | (and) Xanthan Gum | | | |
| | Water | 5.0 | | 1.5 |
| b4) c | Coco-Caprylate | 1.7 | Emollient | 0.51 |
| | Perfume 1 | 0.3 | Perfume oil | 0.09 |
| b4) d | Water, pentylene | 1 | Active biogenic | 0.3 |
| | Glycol, Hamamelis | | ingredient | |
| | Virginiana leaf extract, | | | |
| | Xanthan gum, Caprylyl | | | |
| | Glycol | | | |
| b4) e | Glycerin | 2.0 | Humectant | 0.6 |
| b4) f | D-Panthenol | 2.0 | Active biogenic | 0.6 |
| | | | ingredient | |
| Water tank | Water, demin. | ad 100 | | Make up to 30 g |

All sunscreen fluid formulations produced were homogeneous and stable.

The SPF values of mixtures are reported hereinafter. The SPF values were ascertained using the BASF sunscreen simulator calculation. Mixtures that were produced with capsule b1) and b3) had an SPF of 15. Mixtures that were produced additionally with capsule b2)a had an SPF of 30. Mixtures that were produced additionally with capsule b4)a had an SPF of 30. Mixtures that were produced additionally with capsule b4)b had an SPF of 50. Mixtures that were produced additionally with capsule b2)a and b4)a had an SPF of 50. Mixtures that were produced additionally with capsules b2)a and b4)b had an SPF of 50+. The SPF values of the mixtures comprising b1)+b3) and b1)+b3)+b2)a were likewise tested in vitro. A comparison of the in vitro values with the simulated values shows that the simulation is an excellent approximation to the SPF value (cf. table 13).

### Example 12 Formulation of the Base Mixture

Four different base mixtures b1) were produced.

First of all, the UV filters and emollients and a preservative were mixed (portion A). According to the desired base mixture, this portion A was blended with a solid oil mixture, a pre-emulsion or a liquid oil mixture (portion B). The compositions of the corresponding formulations are reproduced in tables 12a to 12d.

Before the emptying of the base mixture according to table 12a or 12b into the mixing device, it is first heated to about 75° C. in the capsule b1).

TABLE 12a
Base mixtures with solid oil mixture; * this formulation produced can
subsequently be filled into a capsule which is inserted into the machine

| | | | |
|---|---|---|---|
| | Concen- | | |
| | tration | | |
| | in base | | Amount |
| | mixture | | required |
| | in % | | for |
| Ingredient (chem. name) | by wt. | Function | 8.46 g * |

Portion A

| | | | |
|---|---|---|---|
| Ethylhexyl triazone | 5.3 | UV filter | 0.45 |
| Diethylhexyl butamido triazone | 5.3 | UV filter | 0.45 |
| Bis-ethylhexyloxyphenol | 3.5 | UV filter | 0.3 |
| methoxyphenyl triazine | | | |
| Diethylamino hydroxybenzoyl | 10.6 | UV filter | 0.9 |
| hexyl benzoate | | | |
| Di-n-butyl adipate | 17.7 | Emollient | 1.5 |
| Di-n-Octyl carbonate | 17.7 | Emollient | 1.5 |
| Glycerides, mixed coco, | 10.6 | Emollient | 0.9 |
| decanoyl and octanoyl | | | |
| 2-Phenoxyethanol, | 1.8 | Preservative | 0.15 |
| Ethylhexylglycerin | | | |

Portion B

| | | | |
|---|---|---|---|
| Cetylstearyl alcohol | 8.9 | Rheology | 0.75 |
| | | modifier | |
| Disodium Cetearyl Sulfosuccinate | 2.5 | Interface- | 0.21 |
| | | active | |
| | | substance | |
| Cetearyl Glucoside (and) Cetearyl | 7.1 | Interface- | 0.6 |
| Alcohol | | active | |
| | | substance | |
| Myristyl myristate | 8.9 | Emollient | 0.75 |

TABLE 12b
Base mixtures with solid oil mixture ; * this formulation produced can
subsequently be filled into a capsule which is inserted into the machine

| | | | |
|---|---|---|---|
| | Concen- | | |
| | tration | | Amount |
| | in base | | required |
| | mixture in | | for |
| Ingredient (chem. name) | % by wt. | Function | 9.96 g * |

Portion A

| | | | |
|---|---|---|---|
| Ethylhexyl triazone | 4.2 | UV filter | 0.45 |
| Diethylhexyl butamido triazone | 4.2 | UV filter | 0.45 |
| Bis-ethylhexyloxyphenol | 3.1 | UV filter | 0.3 |
| methoxyphenyl triazine | | | |
| Diethylamino hydroxybenzoyl hexyl | 9.0 | UV filter | 0.9 |
| benzoate | | | |
| Di-n-butyl adipate | 15.1 | Emollient | 1.5 |
| Di-n-Octyl carbonate | 15.1 | Emollient | 1.5 |
| Glycerides, mixed coco, decanoyl | 9.0 | Emollient | 0.9 |
| and octanoyl | | | |
| 2-Phenoxyethanol, | 1.5 | Preservative | 0.15 |
| Ethylhexylglycerin | | | |

Portion B

| | | | |
|---|---|---|---|
| Cetylstearyl alcohol | 15.1 | Rheology | 1.5 |
| | | modifier | |
| Disodium Cetearyl Sulfosuccinate | 2.1 | Interface- | 0.21 |
| | | active | |
| | | substance | |
| Cetearyl Glucoside (and) Cetearyl | 6.0 | Interface- | 0.6 |
| Alcohol | | active | |
| | | substance | |
| Myristyl myristate | 15.1 | Emollient | 1.5 |

TABLE 12c
Base mixtures with pre-emulsion; * this formulation produced can
subsequently be filled into a capsule which is inserted into the machine

| | | | |
|---|---|---|---|
| | Concen- | | |
| | tration | | |
| | in base | | Amount |
| | mixture | | required |
| | in % | | for |
| Ingredient (chem. name) | by wt. | Function | 10.25 g * |

Portion A

| | | | |
|---|---|---|---|
| Ethylhexyl triazone | 4.4 | UV filter | 0.45 |
| Diethylhexyl butamido triazone | 4.4 | UV filter | 0.45 |
| Bis-ethylhexyloxyphenol | 2.9 | UV filter | 0.3 |
| methoxyphenyl triazine | | | |
| Diethylamino hydroxybenzoyl hexyl | 8.8 | UV filter | 0.9 |
| benzoate | | | |
| Di-n-butyl adipate | 14.6 | Emollient | 1.5 |
| Di-n-Octyl carbonate | 14.6 | Emollient | 1.5 |
| Glycerides, mixed coco, decanoyl | 8.8 | Emollient | 0.9 |
| and octanoyl | | | |
| 2-Phenoxyethanol, | 1.45 | Preservative | 0.15 |
| Ethylhexylglycerin | | | |

Portion B

| | | | |
|---|---|---|---|
| Sucrose Polystearate (and) Cetyl | 4.4 | Interface- | 0.45 |
| Palmitate | | active | |
| | | substance | |
| Lauryl Glucoside (and) Polyglyceryl- | 4.4 | Interface- | 0.45 |
| 2 Dipolyhydroxystearate | | active | |
| | | substance | |
| Water | 30.7 | | 3.15 |
| Xanthan Gum | 0.44 | Rheology | 0.045 |
| | | modifier | |

TABLE 12d
Base mixtures with liquid oil mixture; * this formulation produced can
subsequently be filled into a capsule which is inserted into the machine

| | | | |
|---|---|---|---|
| | Concen- | | |
| | tration | | |
| | in base | | |
| | mixture | | Amount |
| | in % | | required |
| Ingredient (chem. name) | by wt. | Function | for 6.75 g * |

Portion A

| | | | |
|---|---|---|---|
| Ethylhexyl triazone | 6.67 | UV filter | 0.45 |
| Diethylhexyl butamido triazone | 6.67 | UV filter | 0.45 |
| Bis-ethylhexyloxyphenol | 4.44 | UV filter | 0.3 |
| methoxyphenyl triazine | | | |
| Diethylamino hydroxybenzoyl hexyl | 13.33 | UV filter | 0.9 |
| benzoate | | | |
| Di-n-butyl adipate | 22.22 | Emollient | 1.5 |
| Di-n-Octyl carbonate | 22.22 | Emollient | 1.5 |
| Glycerides, mixed coco, decanoyl | 13.04 | Emollient | 0.9 |
| and octanoyl | | | |
| 2-Phenoxyethanol, | 2.22 | Preservative | 0.15 |
| Ethylhexylglycerin | | | |

Portion B

| | | | |
|---|---|---|---|
| Polyglyceryl-2 | 8.89 | Interface- | 0.6 |
| Dipolyhydroxystearate | | active | |
| | | substance | |

### Example 13

The SPF value of the sunscreens from examples 9 to 11 that were produced according to table 13 were each ascertained in an in vitro measurement as well as the simulation calculations.

The SPF in vitro and UVA-PF tests are based on ISO 24443. The test is based on the ascertaining of UV transmittance through a thin sunscreen film of a sample applied to a roughened substrate -SB6 PMMA sheet (roughness 4 to 5 μm, area 4.8×4.8 cm) from Helioscreen, Paris. The sunscreen formulation that was to be tested was applied to the PMMA sheet in an amount of 1.2 mg/cm². Three sheets and 5 measurements per sheet were examined per formulation. As far as possible, the formulation was applied uniformly and manually to the sheets. The sheets with the formulation applied were stored in a dark area at room temperature for 30 minutes before the measurement was conducted.

### Measurement:

### Step 1

In vitro transmittance measurements were conducted from 290 to 400 nm in 1 nm steps before each UV irradiation with the Labsphere UV Transmittance Analyzer UV 2000S. The initial UV transmission spectrum was recorded, and the SPF in vitro value was calculated before the irradiation (average SPF of all 15 values (of the three sheets and the 5 measurements per sheet)).

### Step 2

A mathematical correction of the initial UV transmission spectrum was undertaken with the coefficient “c” according to ISO 24443, and the UVA-PF in vitro was calculated before the irradiation.

### Step 3

The UV irradiation of the formulation was conducted with the Atlas Suntest CPS+ Solar Simulator with the calculated UV dose according to ISO 24443.

### Step 4

The in vitro transmittance measurement after the UV irradiation and the detection of the UV transmission spectrum after the irradiation were conducted. The in vitro SPF value after the irradiation was found (average of the 15 values from the three sheets and the 5 measurements per sheet).

### Step 5

A mathematical correction of the UV transmission spectrum after the irradiation was undertaken with the same coefficient “c” (as per step 2). The UVA-PF in vitro after the UV irradiation was calculated.

TABLE 13
Comparison of the SPF values that were obtained via simulation
calculation with the SPF values that were obtained via in vitro
experiments; *SPF values greater than 80 cannot be reported
exactly via in vitro measurement since the fluctuations in the
values here become too great

| | | |
|---|---|---|
| | | SPF via in vitro |
| Examples | SPF via simulation | measurement |

7

| | | |
|---|---|---|
| b1) | 15 | 19 |
| b1) + b2)a | 30 | 41 |

8

| | | |
|---|---|---|
| b1) + b2)a + b3)a | 50 | 69 |
| b1) + b2)a + b3)b + b4)d | 50+ | >80* |

9

| | | |
|---|---|---|
| b1)a | 15 | 15 |
| b1)a + b2)a | 20 | 26 |
| b1)a + b2)b | 30 | 47 |
| b1)a + b2)c + b3)a | 50 | >80* |
| b1)b | 15 | 20 |
| b1)b + b2)a | 20 | 28 |
| b1)b + b2)b | 30 | 35 |
| b1)b + b2)c + b3)a | 50 | >80* |

10

| | | |
|---|---|---|
| b1) | 10 | 11 |
| 11 | | |
| b1) + b3) | 15 | 15 |
| b1) + b3) + b2)a | 30 | 37 |

1.-64. (canceled)

65. A method of producing a sunscreen in domestically customary amounts, controllable by and/or according to the wishes of the end user of the sunscreen (“personalization”), in a device having a mixing device a) and at least one dispensing device b) equipped with at least 2 dispensing vessels, in which the base mixture is present in one of the dispensing vessels b1) and the active cosmetic ingredients suitable for personalization in the other of the dispensing vessels b2), in which the contents of the dispensing vessels b1) and b2) are emptied into the mixing device a) and mixed in the mixing device, wherein the contents of the dispensing vessel b1) comprising the base mixture are emptied into the mixing device together with at least one emollient, at least one interface-active substance and at least one UV filter, wherein the contents of the dispensing vessel b1) also comprise at least one rheology modifier or no rheology modifier.

66. The method according to claim 65, wherein the base mixture has an SPF of 6 to 20.

67. The method according to claim 65, wherein the base mixture comprises at least one triazine derivative UV filter and at least one UV-A filter.

68. The method according to claim 65, wherein the base mixture comprises at least one emollient selected from the group formed by esters of C2-C12 dicarboxylic acids with linear or branched alcohols having 1 to 22 carbon atoms, mono- and/or dicarbonates of linear or branched C6-C22 fatty alcohols, hydrocarbons, esters of linear C6-C22 fatty acids with linear or branched C6-C22 fatty alcohols, liquid mono-/di-/triglyceride mixtures based on C₆-C₁₅ fatty acids, vegetable fats and oils, and Guerbet alcohols based on fatty alcohols having 6 to 18 carbon atoms.

69. The method according to claim 65, wherein the base mixture comprises at least one nonionic, anionic, cationic and/or amphoteric interface-active substance.

70. The method according to claim 65, wherein the base mixture comprises at least one rheology modifier.

71. The method according to claim 65, wherein the base mixture has a ratio of emollient:interface-active substance in the range from 1:4 to 25:1 and/or wherein the base mixture comprises a rheology modifier and has a ratio of emollient:interface-active substance+rheology modifier in the range from 1:10 to 10:1.

72. The method according to claim 65, wherein
i) the base mixture is produced by means of a solid oil mixture or a pre-emulsion or
ii) the base mixture is produced by means of a liquid oil mixture.

73. The method according to claim 65, wherein
b1) comprises a base mixture comprising
10% to 60% by wt. of at least one emollient,
1% to 20% by wt. of at least one nonionic interface-active substance,
0% to 15% by wt. of at least one rheology modifier,
5% to 50% by wt. of at least one UV filter,
0% to 25% by wt. of active cosmetic ingredients and/or cosmetic auxiliaries or additives and
0% to 50% by wt. of water,
based in each case on the total weight of the base mixture and under the condition that
b2) comprises at least one UV filter and optionally active cosmetic ingredients for personalization, cosmetic auxiliaries and additives, and emollients, and optionally
b3) and optionally
b4) comprise further UV filters, active cosmetic ingredients for personalization, auxiliaries and additives, and emollients.

74. The method according to claim 65, wherein the dispensing vessel b2) comprises a UV filter-comprising formulation.

75. The method according to claim 65, wherein the dispensing device is equipped with a further dispensing vessel b3) and optionally dispensing vessel b4).

76. The method according to claim 65, wherein the base mixture is produced by means of a liquid oil mixture, and wherein the dispensing device is equipped with a further dispensing vessel b3) and b3) comprises a water-based formulation comprising water, at least one interface-active substance, optionally a rheology modifier, optionally glycerol and optionally a UV filter.

77. The method according to claim 65, wherein the device additionally includes a water tank c), where the contents from dispensing vessels b1) and b2) and optionally b3) and optionally b4) are emptied into the mixing device a) and mixed with the water from the water tank c).

78. The method according to claim 77, wherein the contents from the dispensing vessels b1) and b2) and any b3) and optionally b4) are emptied into the mixing device a) and mixed with water at least in amounts of 5% by weight to 75% by weight—based on the cosmetic formulations—from the water tank c).

79. The method according to claim 65, wherein the mixing in the mixing device a) is conducted in a mixing vessel a1) having a lowerable stirrer at the base of the mixing vessel a1), where the stirrer at the base of the mixing vessel a1) is lowered and the mixing vessel is closed.

80. The method according to claim 65, wherein the dispensing vessels b1), b2) and any b3) and optionally b4), before being emptied into the mixing vessel a1), are heated in a heating system d), preferably to temperatures up to 80° C.

81. The method according to claim 65, wherein the dispensing vessels b1), b2), any b3) and any b4) are capsules.

82. The method according to claim 65, wherein the device is controlled by information or data transmitted electronically to the device or end user, preferably by means of a QR code, and/or
wherein the end user provides the dispensing device b) with the dispensing vessels b1), b2) and any b3) and optionally b4), preferably in accordance with the information or data transmitted electronically to the end user,
and/or
wherein the method temperatures of the heating system for the dispensing vessels and/or the amounts of water are controlled by means of the water tank via information/data on the outer wall of the dispensing vessels.

83. A dispensing vessel comprising a base mixture of at least one emollient, at least one interface-active substance, at least one UV filter and at least one rheology modifier or no rheology modifier for the production of domestically customary amounts of sunscreens that can be produced immediately in situ by or at the wish of the end user and are variable in terms of their composition, for a method according to claim 65.

84. The use of a base mixture in a dispensing vessel comprising at least one emollient, at least one interface-active substance, at least one UV filter and at least one rheology modifier or no rheology modifier in combination with at least one UV filter, preferably at least one water-dispersed UV filter, and/or active cosmetic ingredients for personalization, present in a further dispensing vessel, for the production of domestically customary amounts of sunscreens that can be produced immediately in situ at the wish of the end user, preferably for a method according to claim 65.
